(19) United States
(12) Patent Application Publication
(10) Pub. No.: US 2026/0183029 A1
(43) Pub. Date: Jul. 2, 2026

(54) Reduction Trauma Screws and Related Methods

(71) Applicant: Allumin8, Inc., Springfield, MO (US)

(72) Inventors: Alyssa Huffman, Springfield, MO (US); Matthew Shomper, Xenia, OH (US)

(73) Assignee: Allumin8, Inc., Springfield, MO (US)

(21) Appl. No.: 19/130,981

(22) PCT Filed: Dec. 1, 2023
(86) PCT No.: PCT/US23/82145

§ 371 Date: May 19, 2025

Related U.S. Application Data
(60) Provisional application No. 63/385,682, filed on Dec. 1, 2022.

(51) Int. Cl. A61B 17/86; A61B 17/68

(52) U.S. Cl. CPC A61B 17/8625; A61B 17/8645; A61B 17/866; A61B 2017/681

(57) ABSTRACT

Provided herein are a reduction trauma screw and a method for its implantation. The screw comprises a headless screw with narrow threads at the proximal end and a reduction cap. The cap is adapted to couple to the headless screw via the narrow threads after the screw has been inserted into a substrate, such as a patient's bone. The method involves inserting the headless screw into a bone borehole using a headless driver and coupling a reduction cap to the inserted screw using a reduction driver loaded with the cap. This counter-torque approach allows for effective implantation of the reduction trauma screw. These innovations offer potential improvements in patient care, particularly in the areas of bone reduction, traction, and fixation.

This application claims the benefit of priority of U.S. Provisional Patent Application Ser. No. 63/385,682 filed Dec. 1, 2022, the disclosure of which is incorporated by reference in its entirety for all purposes.

This disclosure relates to a reduction trauma screw and related methods for bone implantation, for example, with plates, for reduction, traction, and fixation.

When a bone fractures, the fragments lose their alignment by displacing or angulating. The bony fragments must be re-aligned to their normal anatomical position for the fractured bone to heal without any deformity. Orthopedic surgery attempts to recreate the fractured bone's normal anatomy by reducing the displacement, a restoration to the bone's natural position.

Reduction reverses the forces that led to the displacement of bone. For example, if an external foot rotation produced the fracture, then reduction applies a moment to produce internal rotation. This moment neutralizes the displacement from the external rotation. Closed reduction is the manipulation of bone fragments without surgical exposure of the fragments. Open reduction is where the fracture fragments are exposed surgically by dissecting the tissues. In either case, fixation can be internal, using plates and screws.

Risks and complications may include bacterial colonization of the bone, infection, stiffness, loss of range of motion, nonunion, mal-union, damage to the muscles, nerve damage and palsy, arthritis, tendonitis, chronic pain associated with plates, screws, and pins, compartment syndrome, deformity, audible popping and snapping, and potential future surgeries to remove the hardware.

Moreover, bone mineral density decreases after medical devices are implanted, regardless of the material used. This loss contributes to common medical device failures, including screw loosening, screw backout, and rod breakage. Though many devices promote fusion in an interbody cage, none have been developed for scaffolding and increasing bone mineral density within the vertebral body. Also, the structure of cortical bone within the vertebrae differs from the bone in other parts of the human body.

These challenges relating to long-term stability, such as bone quality and functional ability to heal, have yet to be satisfied. None of the prior technologies have addressed the top two reasons for implant failure revision surgery: screw backout and rod breakage before the patient achieves fusion.

### SUMMARY

The present disclosure provides a reduction trauma screw. This screw comprises a headless screw with narrow threads at the proximal end and a reduction cap. The cap is adapted to couple to the headless screw via the narrow threads after the headless screw has been inserted into a substrate.

The present disclosure also provides a method of implanting a reduction trauma screw. This method comprises inserting a headless screw with a headless driver into a borehole in a bone. Then, a reduction cap is coupled to the inserted headless screw via a reduction driver loaded with the reduction cap. This coupling is achieved using the headless driver to counter-torque against the reduction driver.

Additional embodiments and features are set forth in part in the following description. They will become apparent to those skilled in the art upon examination of the specification or may be learned by the practice of the embodiments discussed herein. A further understanding of the nature and advantages of certain embodiments may be realized by reference to the remaining portions of the specification and the drawings, which form a part of this disclosure.

### BRIEF DESCRIPTION OF THE DRAWINGS

FIG. 1 shows a front plan view of a headless screw disclosed herein.

FIG. 2 shows a side plan view of a headless screw of FIG. 1.

FIG. 3 shows a top plan view of a headless screw of FIG. 1.

FIG. 4 shows a bottom plan view of a headless screw of FIG. 1.

FIG. 5 shows the top perspective view of a spherical reduction cap.

FIG. 6 shows a cross-sectional view of the headless screw of FIG. 1 engaged with the spherical reduction cap of FIG. 5 implanted into a bone.

FIG. 7 shows the top perspective view of a conical reduction cap with a curved taper.

FIG. 8 shows a cross-sectional view of the headless screw of FIG. 1 engaged with the conical reduction cap of FIG. 7 implanted into a bone.

FIG. 9 shows the top perspective view of a conical reduction cap with a straight taper.

FIG. 10 shows a cross-sectional view of the headless screw of FIG. 1 engaged with the conical reduction cap of FIG. 9 implanted into a bone.

FIG. 11 shows the top perspective view of a conical reduction cap with a straight taper and external threads.

FIG. 12 shows the top perspective view of a flanged reduction cap with external threads.

FIG. 13 shows a cross-sectional view of the headless screw of FIG. 1 engaged with the flanged reduction cap of FIG. 12 implanted into a bone.

FIG. 14 shows the perspective side view of an adapter of a reduction driver for a castellated reduction cap disclosed herein.

FIG. 15 shows the adapter of FIG. 14 coupled to the spherical reduction cap of FIG. 5.

FIG. 16 shows a top perspective view of a polyaxial reduction cap.

FIG. 17 shows a side plan view of the polyaxial reduction cap of FIG. 16.

FIG. 18 shows a cross-sectional view of the polyaxial reduction cap of FIG. 16.

FIG. 19 shows a cross-sectional view of the headless screw of FIG. 1 engaged with the polyaxial reduction cap of FIG. 16 implanted into a bone.

FIG. 20 shows a guidewire inserted down bone.

FIG. 21 shows a drill bit inserted over the guide wire of FIG. 20.

FIG. 22 shows the borehole in the bone after the drill and the guidewire from FIG. 21 are removed.

FIG. 23 shows the insertion of the headless screw of FIG. 1 into the borehole with a headless driver.

FIG. 24 shows a side plan view of an assembly of a reduction cap driver inserted over a headless driver to couple the reduction cap of FIG. 5 to the headless screw of FIG. 1 that was implanted into the borehole, as shown in FIG. 23.

FIG. 25 shows a perspective view of the assembly from FIG. 24.

FIG. 26 shows a perspective view of the bone with the implanted reduction cap visible.

FIG. 27 shows a cross-sectional view of the headless screw of FIG. 1 engaged with the spherical reduction cap of FIG. 5 implanted into a bone.

FIG. 28 shows the top view of the headless screw presenting a folded sheet scaffold.

FIG. 29 shows the bottom view of the headless screw of FIG. 28.

FIG. 30 shows a perspective view of a headless screw of FIG. 28.

FIG. 31 shows the back view of the headless screw of FIG. 28.

FIG. 32 shows the front view of the headless screw of FIG. 28.

FIG. 33 shows a magnified inset of the front view of the headless screw of FIG. 28, highlighting the scaffold.

FIG. 34 shows the top view of the headless screw presenting a folded sheet scaffold and textured threads.

FIG. 35 shows the bottom view of the headless screw of FIG. 34.

FIG. 36 shows a perspective view of a headless screw of FIG. 34.

FIG. 37 shows the back view of the headless screw of FIG. 34.

FIG. 38 shows the front view of the headless screw of FIG. 34.

FIG. 39 shows a magnified inset of the front view of the headless screw of FIG. 34, highlighting the scaffold.

FIG. 40 shows the top view of the headless screw presenting a diamond-structured lattice comprising bone harvesting features at the distal tip and textured threads at the proximal end.

FIG. 41 shows the bottom view of the headless screw of FIG. 40.

FIG. 42 shows a perspective view of a headless screw of FIG. 40.

FIG. 43 shows the back view of the headless screw of FIG. 40.

FIG. 44 shows the front view of the headless screw of FIG. 40.

FIG. 45 shows a magnified inset of the front view of the headless screw of FIG. 40, highlighting the scaffold.

The disclosure will be readily understood by the following detailed description in conjunction with the accompanying drawings, wherein like reference numerals designate like structural elements. The drawings provide exemplary embodiments or aspects of the disclosure and do not limit the scope of the disclosure.

### DETAILED DESCRIPTION

The present disclosure provides a reduction trauma screw. This screw comprises a headless screw with narrow threads at the proximal end and a reduction cap. The cap is adapted to couple to the headless screw via the narrow threads after the headless screw has been inserted into a substrate.

In certain embodiments, the substrate is a bone of a patient in need of reduction, traction, fixation, or combinations thereof.

In certain embodiments, the cap is spherical. In certain embodiments, the cap is conical.

In certain embodiments, the cap is flanged.

In certain embodiments, the cap comprises four flanges radially distributed at 90° intervals.

In certain embodiments, the cap further comprises a collar configured to engage a substrate polyaxially. In certain embodiments, the collar comprises ten flanges radially distributed at 36° intervals.

In certain embodiments, the cap has a curved taper. In certain embodiments, the cap has a straight taper. In certain embodiments, the cap comprises external threads.

In certain embodiments, the cap is conical with a curved taper, conical with a straight taper, conical with a straight taper and external threads, or flanged with external threads.

In certain embodiments, the cap comprises at least one castellation configured to be received by an adapter. In certain embodiments, the cap comprises four castellations radially distributed at 90° intervals. In certain embodiments, each castellation comprises a divot.

In certain embodiments, the reduction trauma screw comprises a scaffold. In certain embodiments, the scaffold is characterized by a randomized porosity pattern typical of a native trabecular bone comprising one or more structural cues that enhance at least one of multipotent mesenchymal stem cell (MSC) differentiation, osteoblast growth, extracellular matrix (ECM) deposition, and new bone formation.

In certain embodiments, the scaffold comprises a triply periodic minimal surface (TPMS) with a cubic repeating pattern defining walls within the scaffold. In certain embodiments, the TPMS is a Schwartz Diamond helically wrapped around a central axis of the screw to define a cubic repeating pattern in X/Y/Z dimensions for the scaffold. In certain embodiments, the TPMS is helically wrapped into a single helix. In certain embodiments, the screw has three radial spokes per turn of the helix. In certain embodiments, the cubic repeating pattern is about 1.8 mm in the X/Y/Z dimensions. In certain embodiments, the helical wrap of the screw is defined with a period three times the size of the cubic repeating pattern. In certain embodiments, the walls are about 0.5 mm thick.

In certain embodiments, the scaffold is a folded sheet scaffold. In certain embodiments, the folded sheet scaffold is a shellular porous pseudorandom orientable architecture comprising a continuous folded sheet of topological genus-n, wherein real 3-dimensional space is divided into disjoint sub-volumes. In certain embodiments, the sub-volumes are incongruent and non-intersecting. In certain embodiments, the pseudorandom architecture is driven by a dimensionless 3-dimensional noise field. In certain embodiments, the sheet architecture has one or more characteristics chosen from continuous, perforated, functionally graded, semi-regular, and driven by modulating algorithms that control spatially-varying features.

In certain embodiments, the threads of the screw near the proximal end are textured with a topography resembling the topography of the scaffold.

In certain embodiments, the screw has reduced one or more screw loosening, screw backout, rod breakage, and lowered bone mineral density.

In certain embodiments, the screw focuses bone growth throughout a shaft to minimize shear stresses on a distal tip and spreads micromotion throughout the screw to encourage bony ingrowth.

In certain embodiments, the screw comprises at least one trephine to harvest bone internally within the screw.

The present disclosure also provides a method of implanting a reduction trauma screw.

This method comprises inserting a headless screw with a headless driver into a borehole in a bone, and coupling a reduction cap to the inserted headless screw via a reduction driver loaded with the reduction cap by using the headless driver to counter-torque against the reduction driver.

In certain embodiments, when the reduction cap is coupled to the headless screw, compression is provided to the bone without moving the headless screw.

In certain embodiments, when present, the collar is adjusted polyaxially before compression is provided to the bone.

In certain embodiments, the method further comprises aligning a drill guide, tissue protector, or both.

In certain embodiments, the method further comprises inserting a guide wire down bone.

In certain embodiments, the method further comprises drilling a borehole with a cannulated drill bit inserted around the guide wire.

In certain embodiments, the method further comprises removing the drill bit and guide wire.

In certain embodiments, the drill bit has a diameter smaller than the screw to be inserted.

In certain embodiments, the drill bit is 3.2 mm in diameter.

In certain embodiments, the method is percutaneous.

### Bone

Bones can generally be divided into cancellous bone and cortical bone. “Cancellous bone,” also called “trabecular bone” or “spongy bone,” is a light, porous bone enclosing numerous large spaces that give a honeycombed or spongy appearance. The bone matrix, or framework, is organized into a three-dimensional latticework of bony processes, called trabeculae, arranged along stress lines. The spaces between are often filled with marrow and blood vessels. In cross-sections, trabeculae of a cancellous bone can look like septa. But, they are topologically distinct in three dimensions, with trabeculae roughly rod or pillar-shaped and septa sheet-like.

Cancellous bone makes up about 20% of the human skeleton, providing structural support and flexibility without compact bone. It is found in most areas of bone not subject to great mechanical stress. It makes up much of the enlarged ends (epiphyses) of the long bones and is the major component of the ribs, the shoulder blades, the flat bones of the skull, and a variety of short, flat bones elsewhere in the skeleton.

Because of the increasing frequency of total joint replacements and their impact on bone remodeling, understanding the stress-related and adaptive process of trabecular bone has become a central concern for bone physiologists. To understand the role of trabecular bone in age-related bone structure and design for the bone-implant system, the mechanical properties of trabecular bone are studied as a function of the anatomic site, density, and age. So, mechanical factors, including modulus, uniaxial strength, and fatigue properties, are also studied.

High porosity makes trabecular bone compliant. Large variations in architecture lead to high heterogeneity. The modulus and strength vary inversely with porosity and highly depend on the porosity structure. Typically, the porosity percent of cancellous bone is between 75% and 95%. The density is between 0.2 and 0.8 g/cm³. Porosity can reduce the strength of the bone but also reduce its weight.

The porosity and its structure affect the strength of the material. Thus, the microstructure of trabecular bone is typically oriented. The “grain” of porosity is aligned where mechanical stiffness and strength are the greatest. Because of the microstructural directionality, the mechanical properties of trabecular bone are highly anisotropic. Young's modulus for trabecular bone is between 800 and 14,000 Mpa. Its strength of failure is 1 to 100 MPa.

“Cortical bone” or “compact bone” is much denser than cancellous bone. It forms the hard exterior (cortex) of bones. The cortical bone gives bone its smooth, white, and solid appearance. It accounts for about 80% of the total bone mass of an adult human skeleton. Cancellous bone is usually surrounded by a shell of compact bone, which provides greater strength and rigidity. The open structure of cancellous bone enables it to dampen sudden stresses, as in load transmission through the joints. Varying proportions of space to bone are found in different bones according to the need for strength or flexibility. Cancellous bone also has a relatively high level of metabolic activity.

“Wolff's law” refers to the bone in a healthy person or animal adapting to the loads under which it is placed. For example, if loading on a particular bone increases, it will remodel itself to become stronger to resist that loading.

### Scaffold

Porous 3D-printed scaffolds promote osseointegration, fusion, and fixation within a bone. The open skeleton with a scaffold is similar to that of native bone. This similarity allows physicians to make patient-specific choices using other agents to promote bone formation and/or stabilize the device.

Triangular-shaped porosity sequences have been used in the prior art. Rounded, square/rectangular shapes and variegated patterns more closely align with native bone structure. In addition, the scaffold's structure reduces the likelihood of revision of the medical device from which it is made, for example, screw loosening, screw backout, rod breakage, and lower bone mineral density.

In certain embodiments, a 3D Voronoi surface lattice structure is applied at the minor diameter of the screw with at least one different lattice size and random shape patterns and sizes. The 3D Voronoi surface lattice structure is defined by a Voronoi diagram, which is a plane partitioned into regions close to each of a given set of objects. In the simplest case, these objects are just finitely many points in the plane (called seeds, sites, or generators). For each seed, the corresponding region is called a “Voronoi cell” or “Thiessen polygon,” consisting of all points of the plane closer to that seed than to any other. The Voronoi diagram of a set of points is dual to that set's Delaunay triangulation.

In certain embodiments, the minor diameter can be constant or variable. In certain embodiments, a plurality of surface lattice structures is superimposed, with each surface lattice structure providing random pore sizes and a different dimensional and/or cross-sectional value for the connecting elements. In certain embodiments, structures of the plurality of surfaces lattice structures are merged and connected, then the interfaces between each structure are rounded and/or blended.

In certain embodiments, the internal lattice structure has a randomized pattern similar to healthy trabecular bone. This natural lattice structure has been described, for example, in Callens et al., “The local and global geometry of trabecular bone” Acta Biomaterialia 130 (2021): 343-361, incorporated herein by reference in its entirety.

In certain embodiments, the average Gaussian curvature distributions of the pores in the scaffold are on hyperbolic (K<0). This prevalence of negative Gaussian curvature is consistent with the high topological complexity (i.e., high genus) of trabecular bone, according to the Gauss-Bonnet theorem. The net curvature captures regions where the trabecular surface is strongly bent without distinguishing between the saddle- or sphere-like nature of these bends. In certain embodiments, the pores comprise arc-like transitions between plate-like elements. In certain embodiments, high net curvature in pores is concentrated in cylindrically shaped rod-like elements.

In certain embodiments, the disclosed scaffold and devices integrate orthopedic products with regenerative medicine to prevent the risks of delayed bone fusion to implanted devices.

In certain embodiments, the scaffold comprises one or more structural cues chosen from porosity, pore size, grain size, and surface topography. Porosity and pore size cue signal mechanical strength, cell settlement, and cell migrations. Grain size cures signal protein absorption, cell adhesion, cell proliferation, and cell adhesion. Surface topography cures signal-specific surface area, cell adhesion, and a material-tissue interface. Other scaffold features include pH and wall thickness. In certain embodiments, the one or more structural cues enhance at least one of multipotent mesenchymal stem cell (MSC) differentiation, osteoblast growth, extracellular matrix (ECM) deposition, and new bone formation. In certain embodiments, the new bone formation occurs after MSC differentiation, osteoblast growth, ECM deposition, or combinations thereof.

In certain embodiments, the scaffold comprises a folded sheet scaffold. As used herein, “folded sheet scaffold” refers to a shellular porous structure with a pseudorandom orientable architecture derived from a continuous folded sheet of a topological genus-n. This architecture divides the three-dimensional space into two distinct, non-intersecting sub-volumes, or labyrinths, which, in certain embodiments, are incongruent. The pseudorandom orientation of this structure is influenced by a dimensionless three-dimensional noise field, the characteristics of which—including type, frequency, jitter, and magnitude—are adjustable. In certain embodiments, the folded sheet scaffold exhibits a continuous, perforated, or functionally graded sheet architecture. In various embodiments, the architecture is semi-regular or determined by specific modulating algorithms that control spatially-varying features.

“Dimensionless” refers to a characteristic, quantity, or property that does not have an associated physical or spatial dimension. It is a measure that is purely numerical and independent of any unit of measurement. In the case of a “dimensionless 3-dimensional noise field,” the term “dimensionless” refers to the noise field being defined or characterized by numerical values that do not correspond to a specific physical dimension but rather serve to influence the properties or characteristics of the folded sheet scaffold.

“Functionally graded” refers to a characteristic of the sheet architecture where its properties vary gradually over volume due to a continuous change in structure or composition. This grading can be designed to meet specific requirements of different parts of the scaffold.

The term “genus-n” is a topological concept referring to the number of “holes” or “handles” in a given surface. In the context of a “continuous folded sheet of topological genus-n,” it denotes the complexity of the folded sheet's structure, with n indicating the number of such features.

“Labyrinth” refers to the complex network-like structure formed within the divided sub-volumes of the three-dimensional space. These labyrinths result from the pseudorandom orientation of the folded sheet scaffold, creating intricate pathways or channels.

“Perforated” refers to the presence of a series of holes or openings in the sheet architecture of the scaffold. These perforations range in size and arrangement. They contribute to the porous nature of the scaffold, influencing its functional properties.

“Semi-regular” refers to a characteristic of the sheet architecture where there is a degree of regularity or consistency in the structure but not absolute uniformity. In certain embodiments, “semi-regular” refers to patterns or features that repeat with some variation.

“Shellular” refers to a specific type of porous structure that resembles a shell or a series of shells. This structure is characteristic of the folded sheet scaffold, contributing to its overall architecture and functional properties.

“Spatially-varying feature” refers to characteristics or properties of the folded sheet scaffold that change or vary across different points or regions in space. These features include, but are not limited to, variations in the structure, composition, or functional properties of the scaffold.

“Statistical variation” in the context of a “dimensionless 3-dimensional noise field” refers to the fluctuations or changes in the noise field that follow a certain statistical distribution. This variation influences the pseudorandom orientation of the folded sheet scaffold.

“Sub-volume” refers to separate or distinct portions of a three-dimensional space that do not share common points or intersect. These sub-volumes are created by dividing the three-dimensional space by the continuous folded sheet of topological genus-n.

In certain embodiments, bone screws were 3D-printed and tested with the diamond lattice structure. In certain embodiments, scoop features are located along a helical pattern, for example, corresponding to the helical pattern of the openings into the internal lattice structure.

The design was based on a triply periodic minimal surface (TPMS), a minimal surface in [symbol] that is invariant under a rank-3 lattice of translations. These surfaces have the symmetries of a crystallographic group. Numerous examples of cubic, tetragonal, rhombohedral, and orthorhombic symmetries are known.

Specifically, a Schwartz Diamond TPMS was used for the lattice, formed from symmetry arguments, remapped from Cartesian coordinates to spherical polar coordinates about the central axis of the screw shaft, sheared to form a helical wrap, thickened, subtracted, and as intersected with the 3D geometry space for the scaffold.

The surfaces were generated using symmetry arguments: Given a solution to Plateau's problem for a polygon, reflections of the surface across the boundary lines also produce valid minimal surfaces that can be continuously joined to the original solution. If a minimal surface meets a plane at right angles, then the mirror image in the plane can also be joined to the surface. Hence, periodic surfaces can be constructed given a suitable initial polygon inscribed in a unit cell.

The equation 1 approximates the TPMS for these bone screws:

cos(x)cos(y)cos(z)−sin(x)sin(y)sin(z)=0 (1)

which is the specific base equation for this embodiment of the bone screw. The x, y, and z variables define the periodicity (i.e., pattern) in X/Y/Z, similar to how cubic lattices are defined. This surface is called a “diamond” because it has two intertwined congruent labyrinths, each having the shape of an inflated tubular version of the diamond bond structure. For the sake of discussion, a regularly repeating cell has been assumed, although TPMS geometry is influenced topologically to be pseudo-random. An exact expression exists regarding elliptic integrals based on the Weierstrass-Ennepar parameterization.

As an equation, it defines the Schwarz D surface through infinite real space. Such a surface splits real space into two identical volumes-Positive space passes into negative space defines the iso- or mid-surface. A 2-dimensional slice through the Schwarz Diamond mathematical field shows “positive” and “negative” space. In certain embodiments, the cubic repeating pattern is 1.8 mm in X/Y/Z.

After creating the Schwarz equation, it was remapped helically (i.e., twisted) to create the base of the final shape. To do this, the equation was mapped from Cartesian space to polar space using conventional methods. The periodicity was mapped cylindrically. That is, the number of “spokes” radially remained a multiple of the selected cell size. The remap was about the central axis of the screw shaft.

After remapping, the space was sheared to create the helical wrap, similar to how an inclined plane is wrapped around a cylinder to create a screw. To create the shear, the Schwarz D equation is remapped from X/Y/Z coordinate space by shearing one (or multiple of the coordinates): x→x, y→y, and z→z+x, where the Schwarz Diamond was sheared in the XZ plane. Such a shear operation maintained a continuous field.

After the field was sheared and remapped cylindrically, it was thickened using an absolute value operation, which converted the negative space of the equation into a positive in three dimensions. The helical wrap of the medical device is defined as a period three times the size of the cubic repeating pattern (5.4 mm), forming a single helix with a circumferential count of three for three radial spokes. After this, a subtract mathematical operation offsets the central geometry to create a sheet-like structure. In certain embodiments, the walls are about 0.50 mm thick. Once the thin-walled lattice field was intersected with the 3D geometry space that defines where the lattice exists, to model for the scaffold was produced. See, for example, FIG. 45.

### Screw

The present disclosure provides a reduction trauma screw comprising a headless screw and a reduction cap.

Screws are the basic elements for achieving interfragmentary compression. They can be used as lag screws, either individually or through plates, to bring two fragments together under compression. Screw are also used to fix plates to the bone. Screw sizes are named according to the outside diameter of their threaded portion. Cortex screws are sized for hard cortical bone, such as in the diaphysis of long bones. Typically, cortex screws are not self-cutting, and the thread has to be cut before insertion. Cancellous bone screws are applied to metaphyseal and epiphyseal regions, where the bone is softer and spongy and the cortex thin.

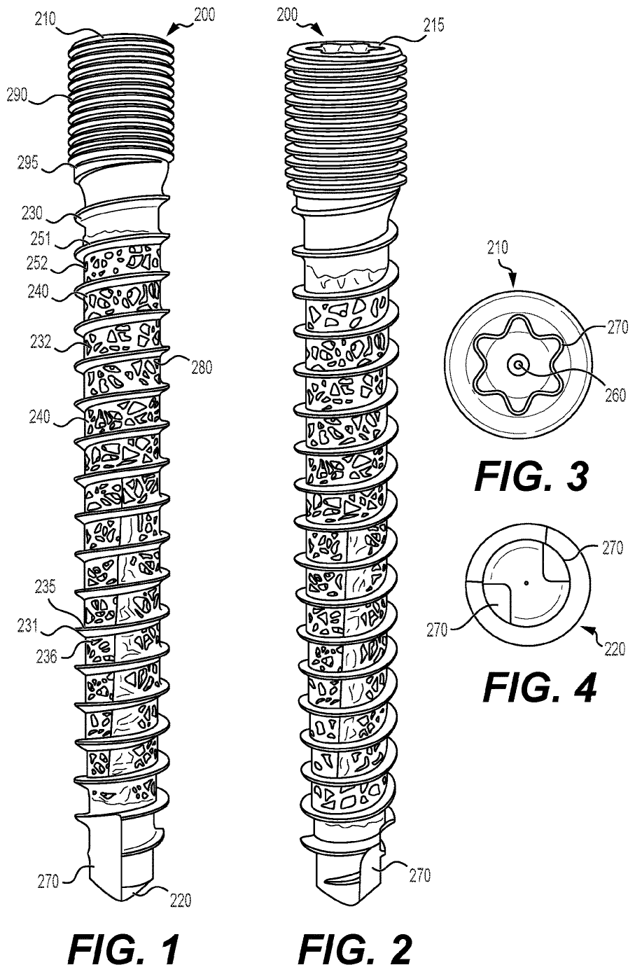

FIG. 1 shows a front plan view of a headless screw 200 disclosed herein. The screw 200 comprises a thread 230 disposed around a core 240 that extends between the proximal end 210 and distal tip 220, and narrow threads 290 disposed on the shaft 295 adapted for receiving a reduction cap 500 at the proximal end 210. The core 240 comprises a scaffold 280 exposed to the outer surface of the screw 200. The thread 230 comprises an external thread form with a leading edge 231 having a leading surface 235 and a trailing edge 232 having a trailing surface 236. The leading surface 235 defines a first opening 251. The trailing surface 236 defines a second opening 252. The first and second openings 251,252 were axially aligned. The screw 200 has a core 240 filled with scaffold 280 extending through the center of the screw 200 from the proximal end 210 to the distal tip 220. The core 240 is the stem of screw 200 from which the threads 230 protrude. The distal tip 220 comprises at least one cutting member 270, each having a cutting edge. The reduction cap 500 is not to be confused with a nut that is sometimes used in emergent situations when the hole for the screw has been stripped. FIG. 2 shows a side plan view of the headless screw 200 of FIG. 1. FIG. 3 shows a top plan view of the headless screw 200 of FIG. 1, including drive 215 and cannula 260. FIG. 4 shows a bottom plan view of the headless screw 200 of FIG. 1, including cutting members 270.

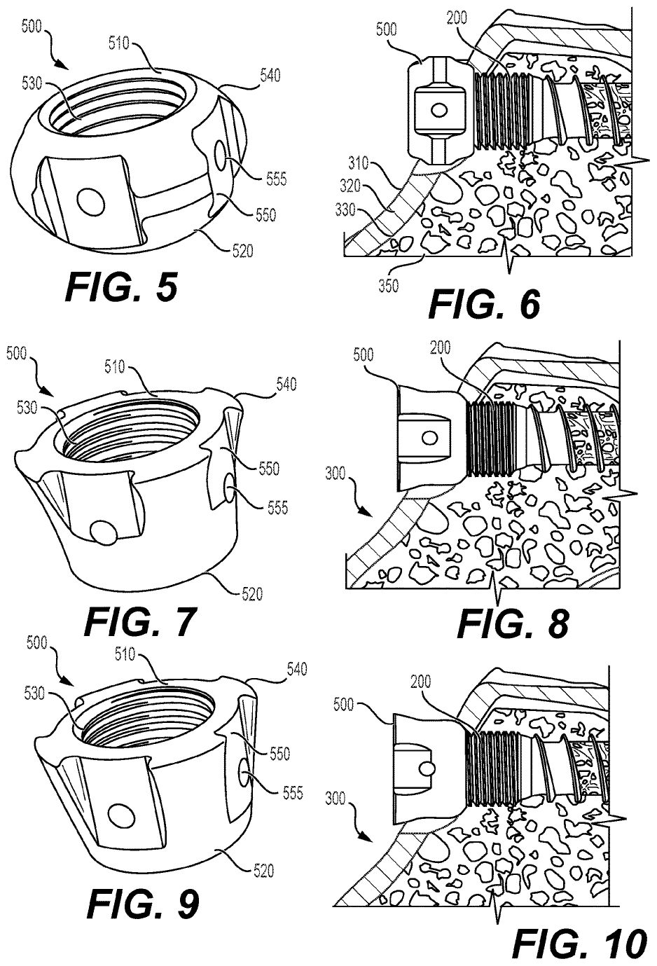

FIG. 5 shows the top perspective view of a spherical reduction cap 500, which can be used as a trauma screw to compress bone or as a compression screw in a plate. The cap 500 comprises a cap body 540 having a cap thread 530 disposed in a spiral around the outer surface of the cap body 540 between the cap top 510 and the cap bottom 520. The body 540 of the cap 500 comprises at least one castellation 550, such as four castellations 550 radially distributed at 90° intervals. The castellation 550 permits the cap 500 to be driven onto a screw 200 that has already been implanted. Each castellation 550 may comprise a divot 555.

FIG. 6 shows a cross-sectional view of the headless screw 200 of FIG. 1 engaged with the spherical reduction cap 500 of FIG. 5 implanted into a bone 300. The screw 200 traverses the cortical bone 320 between the periosteum 310 and the endosteum 330 and into spongy bone 350. The cap 500 compresses the screw 200 by pressing on the cortical bone 320.

FIG. 7 shows the top perspective view of a conical reduction cap 500 with a curved taper, which can be used as a trauma screw to compress bone.

FIG. 8 shows a cross-sectional view of the headless screw 200 of FIG. 1 engaged with the conical reduction cap 500 of FIG. 7 implanted into a bone 300.

FIG. 9 shows the top perspective view of a conical reduction cap 500 with a straight taper, which can be used as a trauma screw to compress bone.

FIG. 10 shows a cross-sectional view of the headless screw 200 of FIG. 1 engaged with the conical reduction cap 500 of FIG. 9 implanted into a bone 300.

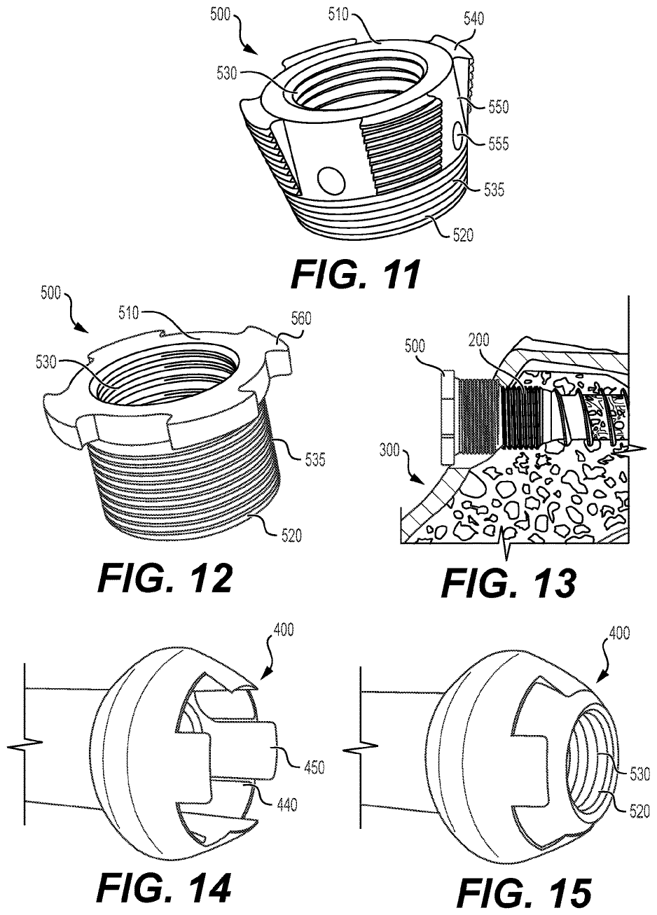

FIG. 11 shows the top perspective view of a conical reduction cap 500 with a straight taper and external threads 535, which can be used as a locking screw in a plate.

FIG. 12 shows the top perspective view of a flanged 560 reduction cap 500 with external threads 535, used as a trauma screw to compress bone or as a compression screw in a plate. In this way, bone strip-out is minimized because the reduction cap creates compression instead of advancing the screw.

FIG. 13 shows a cross-sectional view of the headless screw 200 of FIG. 1 engaged with the flanged reduction cap 500 of FIG. 12 implanted into a bone 300.

FIG. 14 shows the prospective side view of an adapter 400 of a reduction cap driver 900 for a castellated 550 reduction cap 500 disclosed herein. FIG. 15 shows the adapter 400 of FIG. 14 coupled to the spherical reduction cap 500 of FIG. 5. The adapter comprises an opening 440 configured to receive the body 540 of a cap 500 and a plurality of tabs 450 to engage each of a plurality of castellations 550 on the cap 500. The tabs 450 engage the castellations 550 until the cap 500 is installed onto a screw 200.

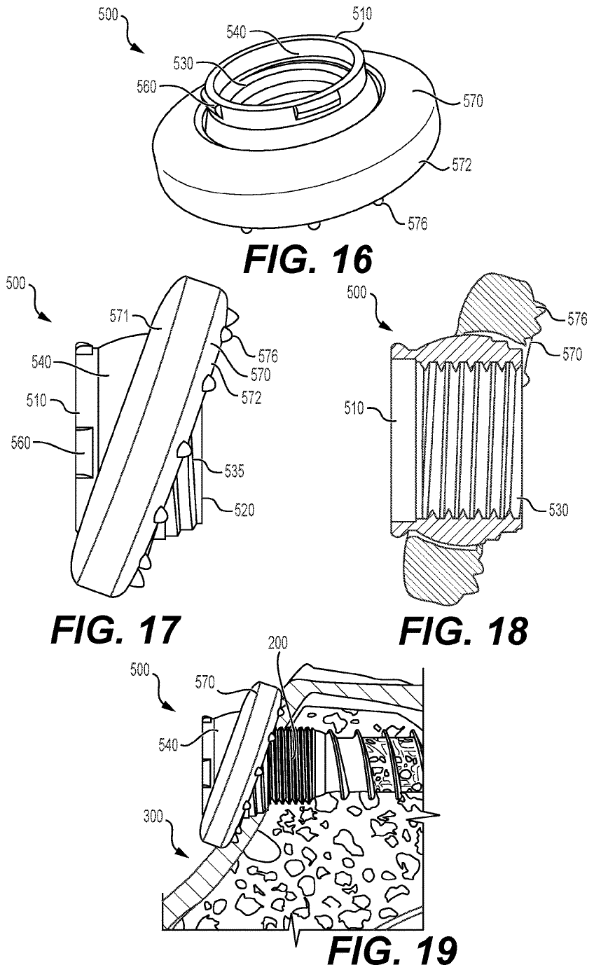

FIG. 16 shows atop perspective view of a polyaxial reduction cap 500, comprising a cap body 540 and a collar 570, having a collar top 571 and a collar bottom 572. The cap body 540 comprises internal threads 530 and flanges 560 radially distributed about the cap top 510. The collar 570 comprises collar flanges 576 radially distributed about the collar bottom 572. The collar 570 is configured to engage a substrate polyaxially.

As used herein, “polyaxial” refers to a characteristic or property of an object or system that allows for rotation or movement along multiple axes. In certain embodiments, a polyaxial system or component can be oriented or adjusted in various directions without being confined to a single plane of rotation. For instance, a polyaxial screw in a surgical application may be adjusted to align with different anatomical structures, or a polyaxial joint in a mechanical system may allow for multi-directional movement.

FIG. 17 shows a side plan view of the polyaxial reduction cap 500 of FIG. 16. In this perspective, one can see the external threads 535 on the cap body 540 near the cap bottom 520.

FIG. 18 shows a cross-sectional view of the polyaxial reduction cap 500 of FIG. 16, highlighting the internal threads 530 on the cap body 540 about the collar 570.

FIG. 19 shows a cross-sectional view of the headless screw 200 of FIG. 1 engaged with the polyaxial reduction cap 500 of FIG. 16 comprising a cap body 540 and a collar 570, and implanted into a bone 300.

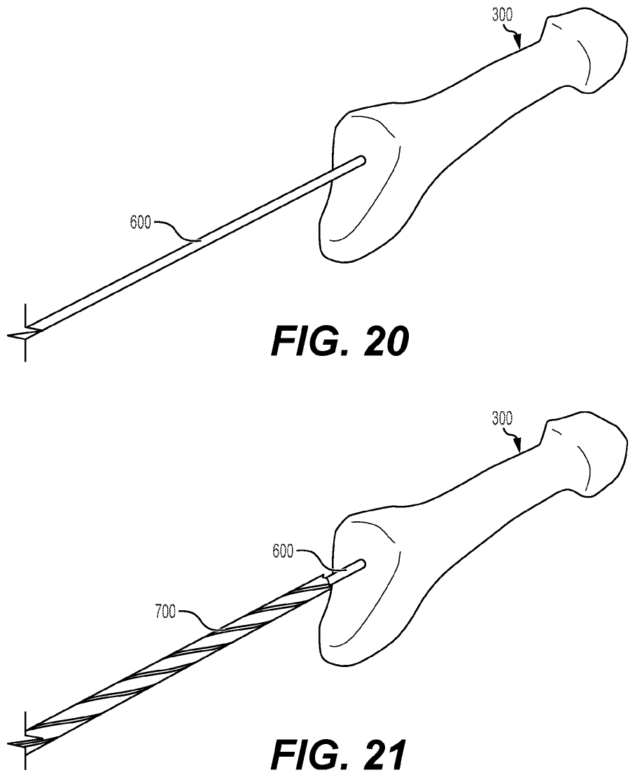

FIG. 20 shows a guidewire 600 inserted down bone 300. The tissue protector is not shown for clarity.

FIG. 21 shows a drill bit 700 inserted over the guidewire 600 of FIG. 20. The tissue protector is not shown for clarity.

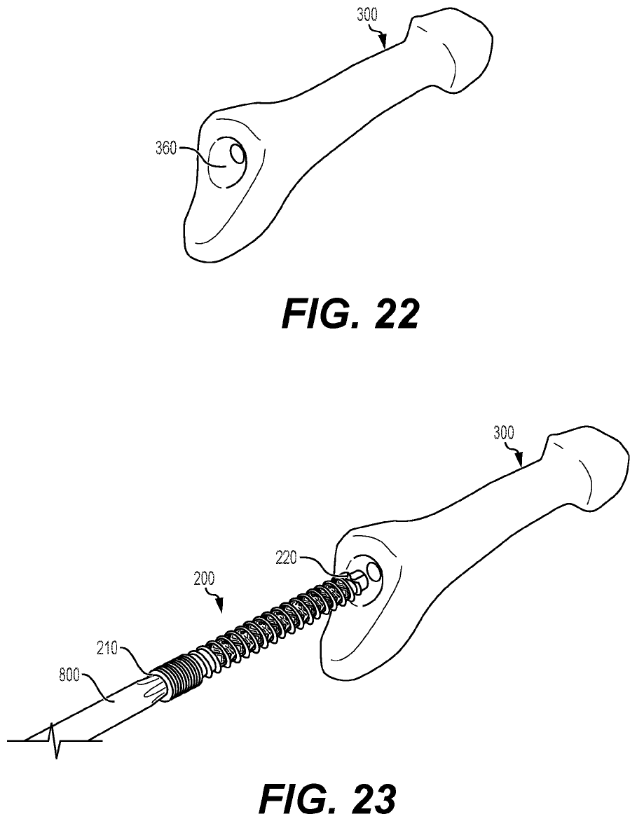

FIG. 22 shows the borehole 360 in the bone 300 after the drill 700, and the guidewire 600 from FIG. 21 are removed.

FIG. 23 shows the insertion of the headless screw 200 of FIG. 1 into the borehole 360 with a headless driver 800.

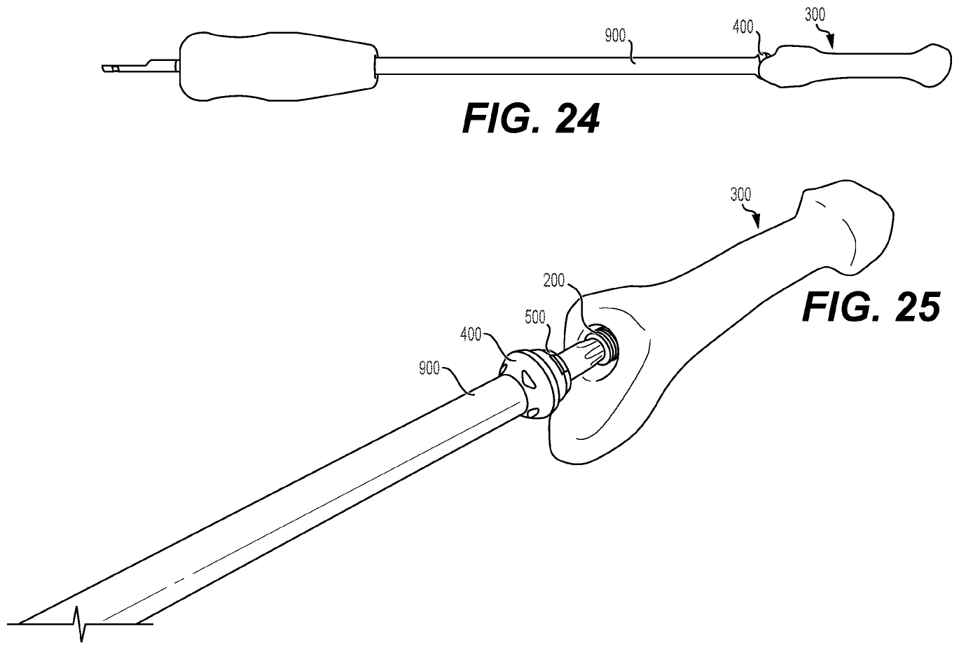

FIG. 24 shows a side plan view of an assembly of a reduction cap driver 900 inserted over a headless driver 800 to couple the reduction cap 500 of FIG. 5 to the headless screw 200 of FIG. 1 that was implanted into the borehole 360, as shown in FIG. 23. An adapter 400 holds the cap 500 for insertion.

FIG. 25 shows a perspective view of the assembly from FIG. 24. The headless driver 800 provides a counter-torque to the reduction cap 500 driver so that compression is achieved between the reduction cap 500 and bone 300 without turning or driving the headless screw 200 further into the bone 300 when the reduction cap driver 900 tightens the cap 500.

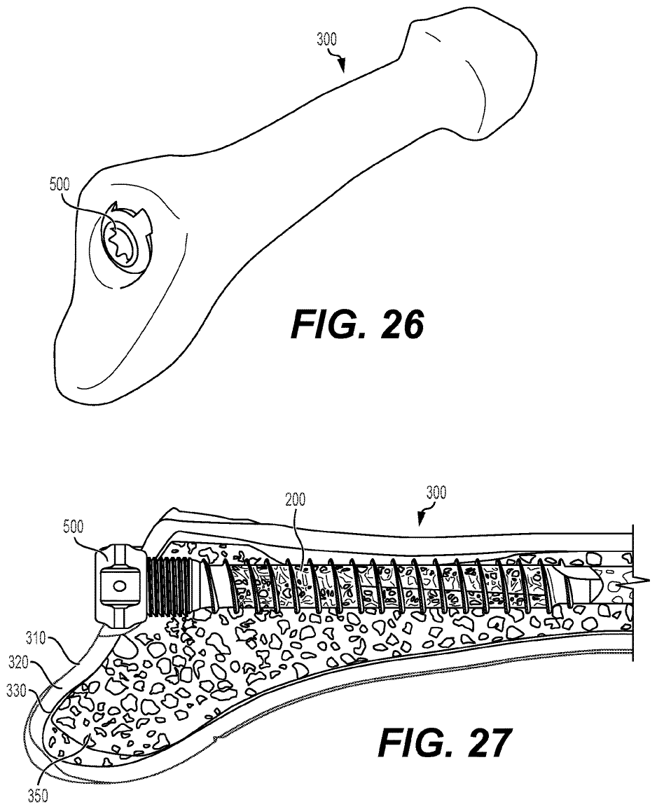

FIG. 26 shows a perspective view of bone 300 with the implanted reduction cap 500 visible.

FIG. 27 shows a cross-sectional view of the headless screw 200 of FIG. 1 engaged with the spherical reduction cap 500 of FIG. 5 implanted into a bone 300.

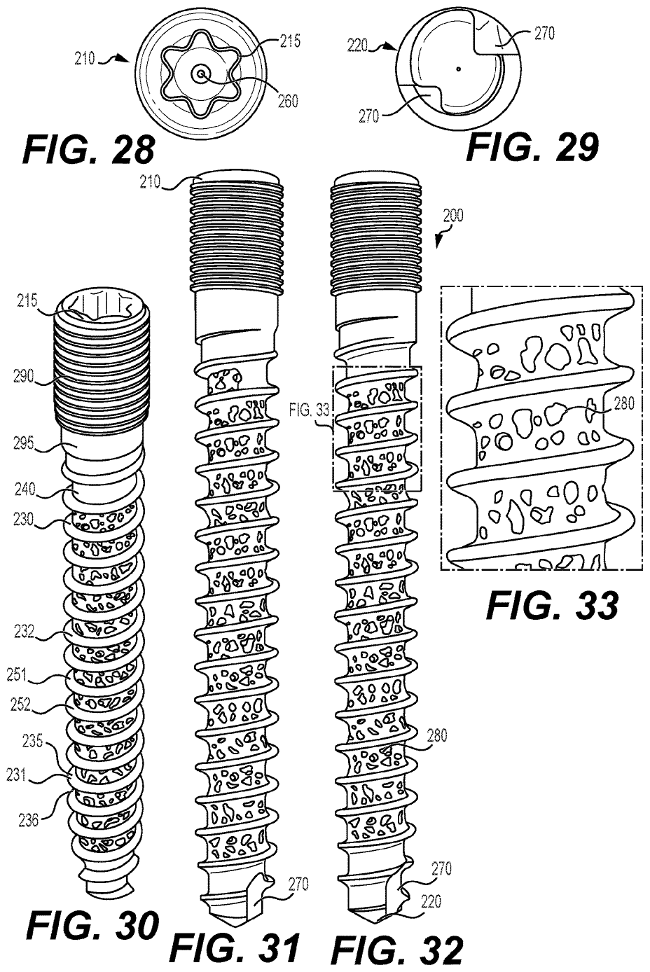

FIG. 28 shows a top plan view of the headless screw 200 presenting a folded sheet scaffold 280. Drive 215 and cannula 260 can be seen from this view. FIG. 29 shows a bottom plan view of the headless screw 200 of FIG. 28, including two cutting members 270. FIG. 30 shows a perspective view, FIG. 31 shows a back plan view, and FIG. 32 shows a front plan view of the headless screw 200 of FIG. 28. FIG. 33 shows a magnified inset of the front view of the headless screw 200 of FIG. 28, highlighting the folded sheet scaffold 280, as described herein.

In this embodiment, the screw 200 comprises a thread 230 disposed around a core 240 that extends between the proximal end 210 and distal tip 220, and narrow threads 290 disposed on the shaft 295 adapted for receiving a reduction cap 500 at the proximal end 210. The core 240 comprises a scaffold 280 exposed to the outer surface of the screw 200. The thread 230 comprises an external thread form with a leading edge 231 having a leading surface 235 and a trailing edge 232 having a trailing surface 236. The leading surface 235 defines a first opening 251. The trailing surface 236 defines a second opening 252. The first and second openings 251,252 were axially aligned.

The screw 200 has a core 240 filled with scaffold 280 extending through the center of the screw 200 from the proximal end 210 to the distal tip 220. The core 240 is the stem of screw 200 from which the threads 230 protrude. The distal tip 220 comprises two cutting members 270 disposed on opposite sides of the distal tip 220, each cutting member 270 having a cutting edge 271.

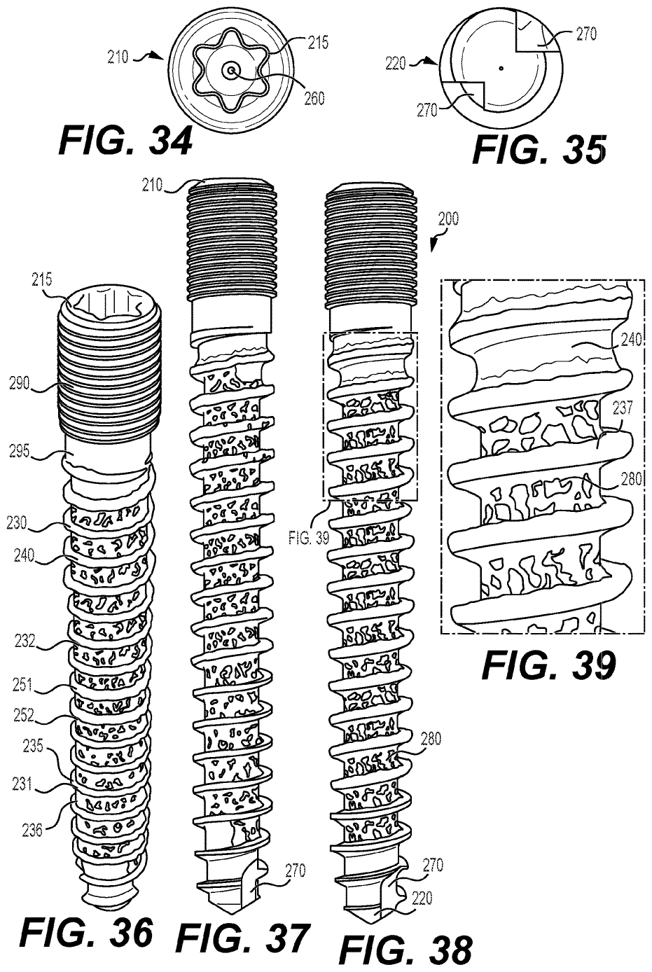

FIG. 34 shows a top plan view of the headless screw 200 presenting a folded sheet scaffold 280. Drive 215 and cannula 260 can be seen from this view. FIG. 35 shows a bottom plan view of the headless screw 200 of FIG. 34, including two cutting members 270. FIG. 36 shows a perspective view, FIG. 37 shows a back plan view and FIG. 38 shows a front plan view of the headless screw 200 of FIG. 34. FIG. 39 shows a magnified inset of the front view of the headless screw 200 of FIG. 34, highlighting the folded sheet scaffold 280, as described herein.

In this embodiment, the screw 200 comprises a thread 230 disposed around a core 240 that extends between the proximal end 210 and distal tip 220, and narrow threads 290 disposed on the shaft 295 adapted for receiving a reduction cap 500 at the proximal end 210. The core 240 comprises a scaffold 280 exposed to the outer surface of the screw 200. The thread 230 comprises an external thread form with a leading edge 231 having a leading surface 235 and a trailing edge 232 having a trailing surface 236. The leading surface 235 defines a first opening 251. The trailing surface 236 defines a second opening 252. The first and second openings 251,252 were axially aligned. The threads 230 near the proximal end 210 are textured 237 to have a surface topography substantially the same as the surface topography of scaffold 280.

The screw 200 has a core 240 filled with scaffold 280 extending through the center of the screw 200 from the proximal end 210 to the distal tip 220. The core 240 is the stem of screw 200 from which the threads 230 protrude. The distal tip 220 comprises two cutting members 270 disposed on opposite sides of the distal tip 220, each cutting member 270 having a cutting edge 271.

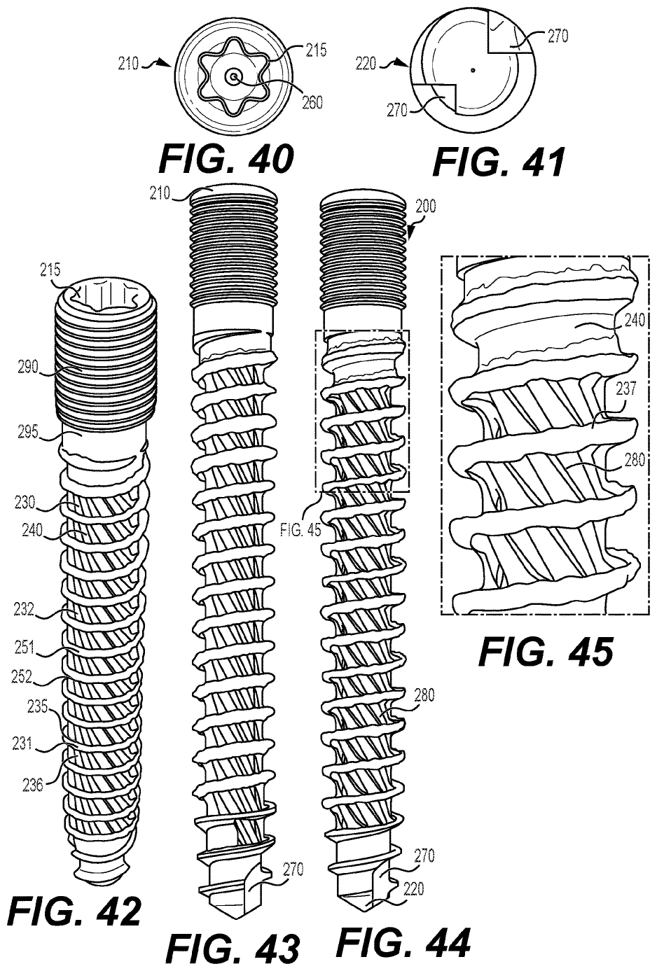

FIG. 40 shows a top plan view of the headless screw 200 presenting a diamond scaffold 280. Drive 215 and cannula 260 can be seen from this view. FIG. 41 shows a bottom plan view of the headless screw 200 of FIG. 40, including two cutting members 270. FIG. 42 shows a perspective view, FIG. 43 shows a back plan view and FIG. 44 shows a front plan view of the headless screw 200 of FIG. 40. FIG. 45 shows a magnified inset of the front view of the headless screw 200 of FIG. 40, highlighting the diamond scaffold 280, as described herein.

In this embodiment, the screw 200 comprises a thread 230 disposed around a core 240 that extends between the proximal end 210 and distal tip 220, and narrow threads 290 disposed on the shaft 295 adapted for receiving a reduction cap 500 at the proximal end 210. The core 240 comprises a scaffold 280 exposed to the outer surface of the screw 200. The thread 230 comprises an external thread form with a leading edge 231 having a leading surface 235 and a trailing edge 232 having a trailing surface 236. The leading surface 235 defines a first opening 251. The trailing surface 236 defines a second opening 252. The first and second openings 251,252 were axially aligned. The threads 230 near the proximal end 210 are textured 237 to have a surface topography substantially the same as the surface topography of scaffold 280.

The screw 200 has a core 240 filled with scaffold 280 extending through the center of the screw 200 from the proximal end 210 to the distal tip 220. The core 240 is the stem of screw 200 from which the threads 230 protrude. The distal tip 220 comprises two cutting members 270 disposed on opposite sides of the distal tip 220, each cutting member 270 having a cutting edge 271.

When present, the pores of scaffold 280 promote boney in-growth through the screw.

In certain embodiments, built-in channels in the screw captured autograft during insertion.

In certain embodiments, the screw is cannulated. In certain embodiments, the screw is non-cannulated. When a fracture in the metaphysis or the epiphysis has been reduced and temporarily fixed with a Kirschner wire, a cannulated screw can be implanted into the site using the Kirschner wire as a guide wire.

The length and diameter of the screw are chosen for the needed application. In certain embodiments, the length is between 8 mm and 200 mm, such as between 34 mm and 60 mm, for example, 34 mm, 36 mm, 38 mm, 40 mm, 42 mm, 44 mm, 46 mm, 48 mm, 50 mm, 52 mm, 54 mm, 56 mm, 58 mm, or 60 mm. In certain embodiments, the length is greater than 8 mm. In certain embodiments, the length is less than 200 mm.

The diameter of the screw can be defined about the thread diameter, the drill bit diameter for a gliding hole or a threaded hole, or the tap diameter. In certain embodiments, the diameter is between 4 mm and 6.5 mm, such as 4.0 mm, 4.5 mm, 5.0 mm, 5.5 mm, 6.0 mm, and 6.5 mm. In certain embodiments, the thread diameter is between 1.0 mm and 7.3 mm, such as 1.0 mm, 1.3 mm, 1.5 mm, 2.0 mm, 2.4 mm, 2.7 mm, 3.0 mm, 3.5 mm, 4.0 mm, 4.5 mm, 6.5 mm, 7.0 mm, 7.1 mm, and 7.3 mm. In certain embodiments, the diameter is greater than 1 mm. In certain embodiments, the diameter is less than 7.3 mm.

In certain embodiments, the screw is self-tapping. In certain embodiments, the screw is non-self-tapping. In certain embodiments, the screw is self-drilling.

The design of the screw thread affects the screw's holding power. Because the strength of the bone is about ten times less than that of the metal of the screw, in certain embodiments, the threads have an asymmetrical buttress profile. For the screw to hold, the threads should engage the entire far cortex of the bone. The tip of the screw and one or two threads should protrude on the opposite of the bone.

In certain embodiments, the thread length of the screw is short. In certain embodiments, the thread length is long. In certain embodiments, the thread length is partial. In certain embodiments, the thread length is full. In certain embodiments, the thread length is a specified length, such as 16 mm or 32 mm.

The drive type for the screw and the cap can be different shapes and sizes, depending on the size of the screw and its application. In certain embodiments, the drive type is cruciform, hexagonal, star, or lobular (Torx). A common example of a cruciform drive type is the Phillips head screw. “Torx” is a trademark for a type of screw drive characterized by a 6-point star-shaped pattern, The official generic name for Torx, standardized by the International Organization for Standardization as ISO 10664, is hexalobular internal.

When a star or lobular drive type, different numbers of points may be used, such as 5-pointed, 6-pointed, 7-pointed, 8-pointed, 10-pointed, or 12-pointed stars or lobular screw drives.

Torx head sizes are described using the capital letter “T” followed by a number ranging from T1 to T100. A smaller number corresponds to a smaller point-to-point dimension of the screw head (the diameter of the circle circumscribed on the cross-section of the tip of the screwdriver). The “external” variants of Torx head sizes are described using the capital letter “E” followed by a number ranging from E4 to E44. See Table 1 for details.

TABLE 1
Properties of various Torx drives

| | Point-to-point distance | | Maximum torque range | | ~E |
|---|---|---|---|---|---|
| Size | (in) | (mm) | (lb · ft) | (N · m) | Torx |
| T1 | 0.035 | 0.90 | 0.015-0.022 | 0.02-0.03 | |
| T2 | 0.039 | 1.00 | 0.052-0.066 | 0.07-0.09 | |
| T3 | 0.047 | 1.20 | 0.10-0.13 | 0.14-0.18 | |
| T4 | 0.053 | 1.35 | 0.16-0.21 | 0.22-0.28 | |
| T5 | 0.059 | 1.50 | 0.32-0.38 | 0.43-0.51 | E2 |
| T6 | 0.069 | 1.75 | 0.55-0.66 | 0.75-0.90 | |
| T7 | 0.083 | 2.10 | 1.0-1.3 | 1.4-1.7 | |
| T8 | 0.094 | 2.40 | 1.6-1.9 | 2.2-2.6 | |
| T9 | 0.102 | 2.60 | 2.1-2.5 | 2.8-3.4 | |
| T10 | 0.110 | 2.80 | 2.7-3.3 | 3.7-4.5 | |
| T15 | 0.132 | 3.35 | 4.7-5.7 | 6.4-7.7 | |
| T20 | 0.156 | 3.95 | 7.7-9.4 | 10.5-12.7 | E4 |
| T25 | 0.177 | 4.50 | 11.7-14.0 | 15.9-19 | E5 |
| T27 | 0.201 | 5.10 | 16.6-19.8 | 22.5-26.9 | |
| T30 | 0.220 | 5.60 | 22.9-27.6 | 31.1-37.4 | E6 |
| T35 | 0.232 | 5.90 | | | E7 |
| T40 | 0.266 | 6.75 | 39.9-48.0 | 54.1-65.1 | E8 |
| T45 | 0.312 | 7.93 | 63.4-76.1 | 86-103.2 | |
| T47 | GM-Style | | | | |
| T50 | 0.352 | 8.95 | 97-117 | 132-158 | E10 |
| T55 | 0.447 | 11.35 | 161-189 | 218-256 | E12 |
| T60 | 0.530 | 13.45 | 280-328 | 379-445 | E16 |
| T70 | 0.618 | 15.70 | 460-520 | 630-700 | E18 |
| T80 | 0.699 | 17.75 | 696-773 | 943-1,048 | E20 |
| T90 | 0.795 | 20.20 | 984-1,094 | 1,334-1,483 | |
| T100 | 0.882 | 22.40 | 1,359-1,511 | 1,843-2,048 | E24 |

In certain embodiments, the drive type is chosen from 1.0 mm cruciform, 1.3 mm cruciform, 1.5 mm cruciform, 2.0 mm cruciform, 2.4 mm cruciform, 3.0 mm cruciform, 2.5 mm hexagonal, 3.5 mm hexagonal, 4.0 mm hexagonal, T8, T15, and T25.

Built-in channels for autograft collection enhance the structural integrity of the implantation. These superiorly resisted bone mineral density loss and reduced micromotion. The randomized porosity pattern of scaffold 280 was typical of native trabecular bone. In addition, built-in struts provided structural integrity.

In certain embodiments, the screw and cap are manufactured in cobalt chrome, titanium, and magnesium-infused titanium. In certain embodiments, the screw and cap comprise Ti-6Al-7Nb, Ti6Al4V-ELI (Grade 5 Titanium Alloy) per ASTM F136, 316L stainless steel per ASTM F138, 316LVM stainless steel per ASTM F138, Mg-PSZ per ASTM F2393-12, or Mg—Ti with 5-35 wt. % Mg. 316L stainless teat typically contains 62.5% iron, 17.6% chromium, 14.5% nickel, 2.8% molybdenum and minor alloy additions. A low carbon content is specified to ensure that the material is free from susceptibility to intergranular corrosion. Titanium alloys have improved biocompatibility, functional performance, excellent corrosion resistance, and no allergic reactions. Other materials fabricating the screw included pre-packed demineralized bone matrix (DBM), pre-packed synthetic DBM, and unpacked DBM.

In certain embodiments, the internal core of the screw is a trephine to collect and harvest autograft upon and/or during insertion of the screw.

In certain embodiments, post-implantation options prevent revision surgery through polymer injection through the screw.

In certain embodiments, the screw does not exhibit screw loosening, screw backout, rod breakage, or lowered bone mineral density.

In certain embodiments, the screw comprises a shaft that is thicker than the core, thereby strengthening the point where rod breakage most frequently occurs during screw installation.

In certain embodiments, the screw has reduced one or more screw loosening, screw backout, rod breakage, and lowered bone mineral density.

The disclosed screws focus on bone growth throughout the core to minimize shear stresses on the distal tip and spread micromotion evenly throughout the screw to encourage bony ingrowth.

In certain embodiments, the scaffold of the screw provides options for simple to complex bone mineral densities and immunocompromised patients. In certain embodiments, the scaffold is impregnated with one or more biologics, antibiotics, demineralized bone matrices, nanotechnology, or regenerative medicine therapies.

In certain embodiments, the screw 200 is configured to help bone in-growth through the screw 200 by using the scaffold 280, similar to native trabecular bone. In combination with the thread 230 and the scaffold 280, the core 240 aids autograft harvesting during the insertion process to push autograft into built-in channels within the core 240 of the screw 200. The walls surrounding the pores harvest the autograft and act as trephines. This structure also aided the screw's 200 structural integrity, resisted bone mineral density loss, and reduced micromotion.

The screws 200 disclosed herein overcome the many failures of the prior art screws. In certain embodiments, the screw lacks a windshield wiper effect. In certain embodiments, the screw resists back out. In certain embodiments, the screw does not exhibit excessive micromotion. In certain embodiments, the screw has a low frequency of low-virulent microorganisms detected by sonication, for example, due to individual screw sterilization and packaging. In certain embodiments, the head and shaft of the screw resist failure. In certain embodiments, the screw is adapted for each type of bone quality. In certain embodiments, the screw has adequate thread depth. In certain embodiments, the screw withstands insertion torque, particularly at the head-to-screw coupling. In certain embodiments, the fatigue lifespan of the screw does not decrease when the screw is fully inserted. In certain embodiments, the screw has good instrumentation. In certain embodiments, the screw achieves angulation for rod acceptance. In certain embodiments, the screw does not have cyclic loading based on physiological conditions during walking. In certain embodiments, the screw does not fail in long-segment posterior cervical fusion, not requiring concomitant C6 or T1 buttress pedicles. In certain embodiments, the screw distributes stress. In certain embodiments, the screw does not immunocompromise the patient. In certain embodiments, the screw does not comprise PEEK. In certain embodiments, the screw does not have tulip or locking cap stresses.

In some embodiments, the distal tip 220 of the screw 200 has a surface configuration chosen from angled, irregular, uniform, non-uniform, offset, staggered, tapered, arcuate, undulating, mesh, porous, semi-porous, dimpled, pointed, textured, or combinations thereof. In some embodiments, the distal tip 220 includes a nail configuration, barbs, expanding elements, raised elements, ribs, and/or spikes to provide a fabrication platform for forming a portion thereon via additive manufacturing. In some embodiments, the distal tip 220 has a cross-section configuration chosen from oval, oblong triangular, square, polygonal, irregular, uniform, non-uniform, offset, staggered, tapered, or combinations thereof.

In some embodiments, the leading surface 235 and/or the trailing surface 236 comprises at least one tissue-gathering member. In some embodiments, the tissue gathering member comprises a cutting edge. In some embodiments, the cutting edge is configured to be rasp-like. In some embodiments, the cutting edge is configured to engage tissue, for example, to cut, shave, shear, incise, or disrupt the tissue. In some embodiments, the cutting edge is configured to be cylindrical, round, oval, oblong, triangular, polygonal, having planar or arcuate side portions, irregular, uniform, non-uniform, consistent, variable, horseshoe shape, U-shape, or kidney bean shape. In some embodiments, the cutting edge is rough, textured, porous, semi-porous, dimpled, knurled, toothed, grooved, or polished to engage and cut the tissue. In some embodiments, the cutting edge forms a tunnel configured to guide, drive, or direct the cut tissue into the void, such as fusing the screw with the tissue.

For example, manipulating the screw 200 by rotation or translation causes the cutting edge 271 of the screw to cut and guide the tissue or bone into the core 240, thereby promoting bone growth and fusion to the screw 200. In some embodiments, the tissue is embedded into the core 240 to promote bone growth and fusion to the screw 200. In some embodiments, the lattice is disposed within the core 240 to form a scaffold 280 for bone growth.

In some embodiments, thread 230 is configured as fine, closely spaced, or shallow to engage with the tissue. In some embodiments, the thread 230 comprises an increased pitch and an equal lead between thread turns. In some embodiments, the thread 230 comprises a smaller pitch or more thread turns per the axial distance to fixate stronger fixation with the tissue or resist loosening from the tissue. In some embodiments, the thread 230 is configured to be continuous along a portion. In some embodiments, the thread 230 is configured to be intermittent, staggered, or discontinuous. In certain embodiments, the thread 230 comprises a single thread turn. In certain embodiments, the thread comprises a plurality of discrete threads.

In some embodiments, the thread 230 comprises a penetrating element, for example, chosen from a nail configuration, barb, expanding element, raised elements, rib, or spike. In some embodiments, the thread 230 is configured as self-tapping or intermittent at the distal tip 220. In some embodiments, the distal tip 220 is rounded. In some embodiments, the distal tip 220 is self-drilling. In some embodiments, the distal tip 220 comprises a solid outer surface.

In certain embodiments, the screw is a 3D-printed porous screw. Its porosity mimics native bone to attach and keep stem cells, growth factors, and other proteins within the structure of the screw and encourage bone growth through the screw, stabilizing the overall construct. During insertion into bone, the built-in trephines collect autograft and regenerative cells within the porous matrix. The disclosed topography attracts bone-forming stem cells within and around the device, reducing overall construct macromotion. In certain embodiments, this device enables surgeons to meet patient-specific needs, such as, but not limited to, spraying/injecting regenerative products to stimulate the bone-forming osteogenic cascade, proactively injecting the screw scaffold with antibiotics for diabetic prone infections, and the option to inject bone cement to further stabilize the construct in severely osteoporotic bone.

In certain embodiments, the screw reduces revision rates, improves bone mineral density, and/or addresses patient-specific needs during spine fusions. In certain embodiments, bone mineral density improves, constructs are stabilized, and the likelihood of revision is reduced.

In certain embodiments, the screw is a 3D-printed titanium porous screw with a porous pattern throughout the screw similar to native bone. Without wishing to be bound by theory, the function of the porous pattern is to attach to the surrounding bone, keeping osteogenic stem cells in place and collecting autograft bone within its porous structure. An advantage of the porous structure is the ability to inject polymers and regenerative therapies through the screw. In certain embodiments, stem cell therapies are injected through the screw implant. In such embodiments, failure likelihood is reduced.

In certain embodiments, the surgeon can inject or spray the screw with autologous concentrated stem cells. Without wishing to be bound by theory, as the screw turns during insertion into the bone, the screw's pores collect an autograft/stem cell mixture internally using its built-in trephines. The osteogenic stem cells then bind with the concentrated blood stem cells and signal the process of mutation and replication, forming more osteogenic cells within the screw, followed by a healing cascade of bone directed within and around the screw. In these embodiments, the combination of (a) osteoconductive (bone grows on the surface), (b) osteoinductive (recruiter of cells for bone healing), and (c) osteogenic (development and formation of bone) healing cascade of the stem cells improve bone mineral density and support superior bone integration and pullout strength.

In certain embodiments, the patient is diabetic and prone to infection. In these embodiments, the surgeon can inject a mixture comprising a calcium sulfate product and antibiotics through the screw before or after insertion or on the screw within the pedicle to provide antibiotic delivery in the area. In certain embodiments, the antibiotics are delivered for between two and six weeks. As such, the likelihood of revision due to infection is reduced.

The present disclosure provides a device formed from a scaffold disclosed herein. In certain embodiments, the device is cannulated and fenestrated with the scaffold. In certain embodiments, the device comprises a threaded distal region, an optionally threaded central region, and an optionally threaded proximal region, depending on the compressive forces.

In some embodiments, the screw is configured with features that facilitate bone to grow through the structure of the screw from opposing sides allowing the bone to connect through the screw. In some embodiments, the structure is narrow, such as through the screw thread, thereby permitting rapid through growth. In some embodiments, the structure is deeper, such as through the minor diameter, thus bonding stronger. In some embodiments, the feature is a void in the screw or porous or structured to promote bone growth. In some embodiments, the structure collects autografts within the channels inside the device. In some embodiments, the feature is impregnated with one or more polymers.

In some embodiments, the device is configured to enhance the stabilization and fixation of bone screws within the bone and improve bone mineral density. In some embodiments, the device includes a spinal implant configured for engagement with cortical bone and cancellous bone. In some embodiments, the device is configured to resist and/or prevent toggling on a bone screw when the bone screw is engaged with dense cortical bone and a less dense cancellous bone resulting from a load on the bone screw. In some embodiments, the device is configured to resist and/or prevent the loosening of the bone screw from the cortical bone and, in some instances, pull it out from the bone. In some embodiments, the device is configured to facilitate bone through-growth to improve bone attachment to the bone screw. In some embodiments, the bone screw is anchored in the bone, thereby reducing pullout. In some embodiments, the bone screw is designed to spread micromotion and reduce shearing to strengthen bone mineral density.

In some embodiments, the device includes a bone screw having bone through-growth through the core of the screw to reduce toggle and potential failure of the screw. In some embodiments, the bone screw includes features that allow the bone to grow through the structure of the bone screw from opposing sides allowing bone to connect through those bone screw structures. In some embodiments, the bone screw includes features that may be narrow, such as through the bone screw thread, which would allow for rapid through-growth. In some embodiments, the bone screw includes features that may be deeper, such as through the minor diameter, which would provide a larger volume of bone through-growth. In some embodiments, the bone screw includes features that may be a void or cavity through opposite sides of the bone screw and/or a void or cavity that enters and exits from the same or adjoining surfaces. In some embodiments, the void or cavity may contain a scaffold for the bone to attach or a porous structure on the surface of the void.

In some embodiments, the bone screw includes features or structures that may be disposed along a core of the bone screw. In some embodiments, the bone screw includes features or structures that may be disposed continuously along a surface of the bone screw, such as, for example, along a distal end. In some embodiments, the bone screw includes features or structures that may be disposed discontinuously along a portion of the bone screw. In some embodiments, the bone screw includes features or structures that may include a scaffold or polymers.

In some embodiments, the device comprises a spinal implant having a hybrid configuration that combines a manufacturing method, such as, for example, one or more prior manufacturing features and materials, and a manufacturing method, such as, for example, one or more additive manufacturing features and materials. In some embodiments, additive manufacturing includes 3-D printing. In some embodiments, additive manufacturing includes fused deposition modeling, selective laser sintering, direct metal laser sintering, selective laser melting, electron beam melting, layered object manufacturing, and stereolithography. In some embodiments, additive manufacturing comprises one or more chosen from rapid prototyping, desktop, direct, digital, instant, and on-demand manufacturing. In some embodiments, the device comprises a spinal implant manufactured by a fully additive process and grown or otherwise printed.

In certain embodiments, the devices comprises one or more chosen from demineralized bone matrix (DBM), pre-packed DBM, pre-packed synthetic DBM, unpacked DBM, and magnesium-infused titanium.

In some embodiments, the device comprises a spinal implant, such as, for example, a bone screw manufactured by combining traditional manufacturing methods and additive manufacturing methods. In some embodiments, the bone screw is manufactured by applying additive manufacturing material, where the bone screw can benefit from the materials and properties of additive manufacturing. In some embodiments, traditional materials are used where the benefits, such as physical properties and cost, are superior to those resulting from additive manufacturing features and materials.

In some embodiments, the device treats a spinal disorder chosen from degenerative disc disease, disc herniation, osteoporosis, spondylolisthesis, stenosis, scoliosis, other curvature abnormalities, kyphosis, tumor, and fractures.

“Treating” or “treatment” of a disease or condition refers to performing a procedure that may include administering one or more drugs to a patient, employing implantable devices, and/or employing instruments that treat the disease, such as microdiscectomy instruments to remove portions bulging or herniated discs, and/or bone spurs, to alleviate signs or symptoms of the disease or condition. Treating or treatment does not require complete alleviation of signs or symptoms, does not require a cure, and specifically includes procedures that have a marginal effect on the patient. For example, treatment can include inhibiting the disease, e.g., arresting its development, or relieving the disease, e.g., causing regression.

“Prevention” refers to alleviation before signs or symptoms of a disease or condition appear. Thus, prevention includes preventing the disease from occurring in a patient who may be predisposed to the disease but has not yet been diagnosed as having it.

“Tissue” includes soft tissue, ligaments, tendons, cartilage, and/or bone. In certain embodiments, the tissue is cancellous bone, cortical bone, or corticocancellous bone.

In some embodiments, devices are used with other osteal and bone-related applications, including diagnostics and therapeutics. In some embodiments, devices are alternatively employed in surgical treatment with a patient in a prone or supine position and/or employ various surgical approaches to the spine, including anterior, posterior, posterior mid-line, lateral, posterolateral, and/or anterolateral approaches, and in other body regions such as maxillofacial and extremities. The devices may also be alternatively employed with procedures for treating the lumbar, cervical, thoracic, sacral, and pelvic regions of a spinal column. The devices may also be used on animals, bone models, and other non-living substrates, for example, in training, testing, and demonstration.

In certain embodiments, the device is a custom medical device. In certain embodiments, the device is adapted for sports medicine.

In certain embodiments, the device is temperature-sensing. In certain embodiments, the device is pH-balancing.

In certain embodiments, the devices are fabricated having a porosity with a porogen that is spheroidal, cuboidal, rectangular, elongated, tubular, fibrous, disc-shaped, platelet-shaped, polygonal, or a mixture thereof. In some embodiments, the porosity is based on a plurality of macropores, micropores, nanopores structures, and/or a combination thereof.

In certain embodiments, the device is fabricated from biologically acceptable materials suitable for medical applications, including metals, synthetic polymers, ceramics, bone material, and composites thereof. In certain embodiments, the device comprises one or more chosen from a metal, ceramic, rubber, hydrogel, rigid polymer, fabric, bone material, and composites thereof.

In certain embodiments, the device comprises a metal chosen from stainless steel alloys, aluminum, commercially pure titanium, titanium alloys, Grade 5 titanium, superelastic titanium alloys, magnesium-infused titanium, cobalt-chrome alloys, superelastic metallic alloys such as nitinol, super elastoplastic metals such as Gum Metal®. In certain embodiments, the device comprises a ceramic and composites thereof, such as calcium phosphate (e.g., Skelite™). In certain embodiments, the device comprises a rubber chosen from polyaryletherketone (PAEK), polyetheretherketone (PEEK), polyetherketoneketone (PEKK), polyetherketone (PEK), carbon-PEEK composites, PEEK-BaSO₄ rubber, polyethylene terephthalate (PET), silicone, polyurethane, silicone-polyurethane copolymer, and polyolefin rubber. In certain embodiments, the device comprises a hydrogel. In certain embodiments, the device comprises fabric. In certain embodiments, the device comprises a rigid polymer chosen from polyphenylene, polyimide, polyetherimide, polyethylene, and epoxy. In certain embodiments, the device comprises bone material chosen from autograft, allograft, xenograft, or transgenic cortical and/or corticocancellous bone. In certain embodiments, the device comprises tissue growth or differentiation factors. In certain embodiments, the device comprises resorbable materials, such as composites of metals and calcium-based ceramics, composites of PEEK and calcium-based ceramics, composites of PEEK with resorbable polymers, totally resorbable materials, such as calcium-based ceramics, for example, calcium phosphate, tri-calcium phosphate (TCP), hydroxyapatite (HA)-TCP, calcium sulfate, or other resorbable polymers, such as polyketide, polyglycolide, polytyrosine carbonate, polycaprolactone, and other combinations.

In certain embodiments, the device comprises a rubber chosen from polyaryletherketone (PAEK), polyetheretherketone (PEEK), polyetherketoneketone (PEKK), polyetherketone (PEK), carbon-PEEK composites, PEEK-BaSO₄ rubber, polyethylene terephthalate (PET), silicone, polyurethane, silicone-polyurethane copolymer, polyolefin rubber, synthetic collagen, and collagen matrix. In certain embodiments, the device comprises synthetic collagen. In certain embodiments, the device comprises a collagen matrix.

In certain embodiments, the device comprises magnesium, vitamins, and minerals. “Vitamin” refers to an organic molecule (or a set of molecules closely related chemically, i.e., vitamers) that is an essential micronutrient that an organism needs in small quantities for the proper functioning of its metabolism. Some sources list fourteen vitamins, including choline, but major health organizations typically list thirteen: vitamin A (as all-trans-retinol, all-trans-retinyl-esters, as well as all-trans-beta-carotene and other provitamin A carotenoids), vitamin B₁ (thiamine), vitamin B₂ (riboflavin), vitamin B₃ (niacin), vitamin B₅ (pantothenic acid), vitamin B₆ (pyridoxine), vitamin B₇ (biotin), vitamin B₉ (folic acid or folate), vitamin B₁₂ (cobalamins), vitamin C (ascorbic acid), vitamin D (calciferols), vitamin E (tocopherols and tocotrienols), and vitamin K (phylloquinone and menaquinones). In the context of nutrition, a “mineral” refers to a chemical element required as an essential nutrient by organisms to perform functions necessary for life, including potassium, chlorine, sodium, calcium, phosphorous, magnesium, iron, zinc, manganese, copper, iodine, chromium, molybdenum, selenium, and cobalt.

In certain embodiments, the device comprises a metal chosen from iron, stainless steel alloys, aluminum, commercially pure titanium, titanium alloys, Grade 5 titanium, superelastic titanium alloys, magnesium-infused titanium, cobalt-chrome alloys, superelastic metallic alloys such as nitinol, super elastoplastic metals such as Gum Metal®. In certain embodiments, the device comprises titanium. In certain embodiments, the device comprises iron.

In certain embodiments, the device is manufactured or 3D-printed from materials such as titanium, titanium alloy, cobalt chrome, carbon fiber, magnesium-infused titanium, iron, or stainless steel. In certain embodiments, the device is manufactured from a shape memory alloy or shape memory polymer, allowing the device to conform to an anatomical shape of the patient's body.

In certain embodiments, the device comprises magnesium-infused titanium. In certain embodiments, the device comprises an angiotensin receptor blocker coating. In certain embodiments, the device comprises a type-1 cartilage collagen coating. In certain embodiments, the device is infused with an antibiotic.

In certain embodiments, the device is used with surgical methods or techniques, including, but not limited to, open surgery, mini-open surgery, minimally invasive surgery (MIS), and percutaneous surgical implantation, whereby the injured bone is accessed through a mini-incision or a sleeve provides a protected passageway to the area. Once access to the surgical site is obtained, surgical treatment can treat a disease or disorder, such as a reduction, traction, or installing plates and screws.

In certain embodiments, the surface of the devices comprises a non-solid configuration, such as a lattice. In some embodiments, the non-solid configuration comprises a porous structure or a trabecular configuration.

In various embodiments, the non-solid configuration is configured to provide one or a plurality of pathways to aid bone growth within and through from one surface to an opposite surface of the device. In some embodiments, the lattice comprises one or more portions, layers, or substrates. In some embodiments, one or more portions, layers, or substrates of the lattice are disposed side by side, offset, staggered, stepped, tapered, end to end, spaced apart, in series, or parallel. In some embodiments, the lattice defines a thickness, which may be uniform, undulating, tapered, increasing, decreasing, variable, offset, stepped, arcuate, angled, and/or staggered. In some embodiments, one or more lattice layers are disposed in a side-by-side, parallel orientation within a wall. In certain embodiments, the lattice comprises one or more layers of a material matrix.

In some embodiments, the lattice comprises a plurality of nodes and openings disposed in rows and columns or randomly. In some embodiments, the plurality of nodes and openings are disposed in series. In some embodiments, the plurality of nodes and openings are disposed in parallel.

In some embodiments, the lattice forms a rasp-like configuration. In some embodiments, the lattice is configured to engage tissue. In certain embodiments, the engagement of the lattice is to cut, shave, shear, incise or disrupt the tissue. In some embodiments, the lattice comprises a configuration chosen from cylindrical, round, oval, oblong, triangular, polygonal having planar or arcuate side portions, irregular, uniform, non-uniform, consistent, variable, horseshoe shape, U-shape, or kidney bean shape. In some embodiments, the lattice is rough, textured, porous, semi-porous, dimpled, knurled, toothed, grooved, or polished, for example, to engage and cut the tissue. In some embodiments, the lattice forms a tunnel configured to guide, drive, or direct the cut tissue into an opening, such as fusing the device to the tissue.

In certain embodiments, the screw 200 is injected or sprayed with a material, such as BMA concentrate, calcium phosphate, biologic, and/or antibiotics. The filled or coated screw rested for 10-15 minutes before insertion for the material to absorb.

In certain embodiments, an aperture in the proximal portion of the screw is configured for a syringe to pull or push cells into the screw structure pre- or post-implantation. In certain embodiments, an adapter connects the syringe to the screw. In certain embodiments, the aperture may be in fluid communication with the porous scaffold of the screw and/or one or more lumina.

In certain embodiments, the screw comprises one lumen in fluid communication with the aperture and extends the length of the screw toward the screw tip.

### Plates

In certain embodiments, screws and caps disclosed herein are used with one or more plates having a plurality of holes to receive the screws and/or caps for fixation of the plate to a bone. Plates may be applied in various modes, including protection (neutralization), compression, bridging, and buttress (antiglide). The plate may be larger or smaller, thicker, or thinner as appropriate to various anatomic sites and loads. The holes in the plate are designed, for example, to receive locking screws or nonlocking screws or to aid dynamic compression.

Open Reduction Internal Fixation (ORIF) involves the implementation of implants to guide the healing process of a bone, as well as the open reduction, or setting, of the bone. Open reduction refers to open surgery to set bones, as is necessary for some fractures. Internal fixation refers to the fixation of screws and/or plates, intramedullary rods, and other devices to enable or facilitate healing. Rigid fixation prevents micro-motion across lines of fracture to enable healing and prevent infection, which happens when implants such as plates (e.g., dynamic compression plate) are used. ORIF techniques are often used in cases involving severe fractures, such as comminuted or displaced fractures, or where the bone would not heal correctly with casting or splinting alone.

The plate fits the shape of the bone. For example, the midshaft of many long bones is straight, so plates applied to these regions are not contoured. However, many bones flare towards their metaphysis, so plates applied in these regions are contoured. A flexible template aids plate contouring. Some plates reduce contact areas on the bone. Reconstruction plates contour easily in complex anatomic locations.

Anatomic plates are pre-contoured to fit the region. These plates are for an average patient, so adjustments may be needed to fit individual patients. A protection plate neutralizes bending and rotational forces to protect a lag screw fixation, whether locking or nonlocking screws are used. For implantation, the fracture is reduced and fixed with one or more lag screws. The appropriately contoured plate is applied to the bone. Screws are inserted in a neutral mode. Depending on the plate design, bone quality, implant availability, and surgeon's preference, fixed angle locking head screws, variable angle locking head screws, or nonlocking screws may be inserted. Every hole need not be filled if enough screws are inserted to obtain sufficient hold to maintain the reduction until the fracture heals.

Compression plates provide stability at fracture sites. If possible, the fracture is reduced and temporarily fixed with clamps. Typically, compression plating is used in transverse and short oblique fractures (<30°). Fracture stability from interfragmentary compression results in direct bone healing. In certain embodiments, self-compressing plates, such as dynamic compression plates, limited contact dynamic compression plates, or limited contact plates, or axial compression results from eccentric screw (load screw) insertion.

In certain embodiments, an articulated tension device provides mechanical compression or distraction before fixation with screws inserted in a neutral mode. As in other procedures, the fracture is reduced approximately and securely attaches a plate to one fragment. The device is anchored to the bone with a screw inserted through the articulated footplate. The hook on the device is inserted into the hole at the end of the plate. As the tensioning screw is then tightened, the two limbs of the device are pulled together, achieving compression at the fracture site. In oblique fractures, the plate creates an axilla following the same principle as prebent dynamic compression plates.

Bridge plating is used for multifragmentary long bone fractures where intramedullary nailing or conventional plate fixation, such as compression or protective plating, is unsuitable. The plate provides relative stability by fixation of the two main fragments, achieving correct length, alignment, and rotation. The fracture site is left undisturbed. Callus formation promotes fracture healing.

Bridge plates, like other plates, are often inserted through a minimally invasive approach to leave the fracture site as undisturbed as possible. Screws are either inserted through a limited approach, only exposing the plate sufficiently for screw insertion, or through small stab incisions. The least surgical disturbance to the fracture site occurs when a minimally invasive percutaneous technique is used for plate insertion. Especially with multifragmentary fractures, using an external fixator or distractor can provide alignment and temporary stability for bridge plating without disturbing the soft tissues at the fracture zone. Proximal and distal pins are inserted carefully to not interfere with the later plating procedure.

Long plates with a long working length allow the distribution of bending stresses over a long plate segment, and the stress per unit area is correspondingly low. This prevents high stress over the fracture site and reduces the risk of plate failure. Long plates also allow for a long lever arm, reducing the risk of screw pullout.

Buttress plates are often used to supplement lag screw fixation of metaphyseal shear or split fractures into the metaphyseal regions. The lag screws may be inserted either through or outside the buttress plate. The fracture is reduced and fixed with one or more lag screws following the standard technique.

In certain embodiments, a washer is used, for example, with osteoporotic bone. When used, the washer has a flat side, which rests on the bone, and a counter sunk side which accepts the screw head of screw cap. The washer prevents the screw form breaking through the thin cortex in the metaphysis and epiphysis by spreading the load over a larger area.

Where locking screws are used, such as those described herein, the bone-plate construct remains stable even if the plate does not directly contact the bone. Therefore, contouring does not need to be as accurate.

Either conventional or locking head screws may be used. When using a nonlocking plate and screws, the plate is precisely adapted to the bone; otherwise, the tightening of the screws may lead to loss of reduction. In nonlocking plate systems, screw loosening may also lead to loss of reduction.

The locking head screws described herein provide more stability in osteoporotic bone by reducing the risk of screw pullout and over-tightening of the screws. Well-reduced fractures stay reduced. In certain embodiments, the screw is unicortical, engaging only one cortex of the bone. In certain embodiments, the screw is bicortical, engaging both cortices of the bone. The plate does not need to be perfectly contoured to the bone. The plate is not pressed against the bone, so the periosteum is not compromised.

Screws are unlikely to loosen from the plate. Similarly, if a bone graft is screwed to the plate, a locking head screw will not loosen during graft incorporation and healing. A locking plate/screw system decreases the risk of inflammatory complications due to hardware loosening. Locking plate/screw systems provide more stable fixation than conventional nonlocking plate/screw systems.

Locking head screws are engaged in the plate, and the plate is not pressed against the bone. This reduces interference to the blood supply to the bone underneath the plate. The plate and screws provide adequate rigidity and do not depend on the underlying bone buttressing (load-bearing osteosynthesis). On each side of the fracture, the screws are locked into the plate and the bone. The result is a rigid frame construct with high mechanical stability (internal-external fixator).

When using a locking plate/screw system, the plate does not have to be precisely adapted to the bone. When tightening a locking head screw, the screw does not cause a direct loss of reduction as it tightens into the threaded plate hole and does not draw the bone fragments to the plate. In a locking system, the screw rarely loosens because the screw head is locked to the plate.

### Manufacturing

The devices disclosed herein can be manufactured using various methods. In some embodiments, manufacturing comprises machining, such as subtractive, deformative, or transformative manufacturing. In some embodiments, manufacturing includes cutting, grinding, rolling, forming, molding, casting, forging, extruding, whirling, grinding, cold working, or combinations thereof. In some embodiments, manufacturing includes a portion of the device formed by a medical machining process. In some embodiments, machining uses computer numerical control (CNC) high-speed milling machines, Swiss machining devices, CNC turning with living tooling, wire EDM 4th axis, and combinations thereof. In some embodiments, the manufacturing for fabricating a portion of the devices includes a finishing process, such as laser marking, tumble blasting, bead blasting, micro blasting, powder blasting, or combinations thereof.

In certain embodiments, the device is fabricated per instructions from a computer and processor based on the digital rendering and/or data of a selected configuration via additive manufacturing.

In some embodiments, additive manufacturing comprises 3-D printing. In some embodiments, additive manufacturing is chosen from fused deposition modeling, selective laser sintering, direct metal laser sintering, selective laser melting, electron beam melting, layered object manufacturing, stereolithography, and combinations thereof. In some embodiments, additive manufacturing comprises rapid prototyping, desktop manufacturing, direct manufacturing, direct digital manufacturing, digital fabrication, instant manufacturing, on-demand manufacturing, or combinations thereof.

In some embodiments, a portion of the device is manufactured by additive manufacturing and then mechanically attached to a surface of the device, for example, by welding, threading, adhesives, or staking.

In one embodiment, the device is configured based on imaging from the patient's anatomy. Suitable imaging techniques include, but are not limited to, X-ray, fluoroscopy, computed tomography (CT), magnetic resonance imaging (MRI), surgical navigation, bone density (DEXA), or acquirable 2-D or 3-D images of patient anatomy. Selected configuration parameters for the device are collected, calculated, or determined. Examples of configuration parameters include, but are not limited to, patient anatomy imaging, surgical treatment, historical patient data, statistical data, treatment algorithms, implant material, implant dimensions, porosity, and manufacturing method. In some embodiments, the configuration parameters comprise implant material and device porosity based on patient anatomy and surgical treatment. In some embodiments, porosity is selected. In some embodiments, the configuration parameter of the device is patient-specific. In some embodiments, the configuration parameter of the device is based on a generic configuration and is not patient-specific.

For example, a digital rendering or data of a device is generated for display from a graphical user interface or storage on a database attached to a computer and a processor. In some embodiments, the computer display via a monitor saves, digitally manipulates, or prints a hard copy of the digital rendering or data. In some embodiments, the device is designed virtually with a CAD/CAM program on a computer display. In some embodiments, the processor executes code stored in a computer-readable memory medium to execute one or more computer instructions, for example, transmitting instructions to an additive manufacturing device. In some embodiments, the database or computer-readable medium comprises RAM, ROM, EPROM, magnetic, optical, digital, electromagnetic, flash drive, semiconductor technology, or combinations thereof. In some embodiments, the processor instructs motors to control the movement and rotation of device components.

The screw is tested in cobalt chrome and meets American Society for Testing and Materials (ASTM) standard 543. ASTM standard 543 evaluates plastic materials for resistance to chemical reagents, including cast, hot-molded, cold-molded, laminated resinous products, and sheet materials. Three procedures are presented, two under practice A (Immersion Test) and one under practice B (Mechanical Stress and Reagent Exposure under Standardized Conditions of Applied Strain). These practices report changes in weight, dimensions, appearance, color, strength, and other mechanical properties. Standard reagents are specified to establish results on a comparable basis without precluding other chemical reagents pertinent to specific chemical resistance requirements. Provisions are made for various exposure times, stress conditions, and exposure to reagents at elevated temperatures. The type of conditioning (immersion or wet patch/wipe method) depends upon the material's end-use.

In certain embodiments, the screw 200 is individually packaged in a double Tyvek™ peel tray.

### Method of Implantation

The present disclosure provides a method of implanting a reduction trauma screw comprising inserting a headless screw with a headless driver into a borehole in a bone and coupling a reduction cap to the inserted headless screw via a reduction driver loaded with the reduction cap.

In certain embodiments, the method of implantation further comprises aligning a drill guide and/or tissue protector.

In certain embodiments, the method of implanting further comprises inserting a guidewire down bone. In certain embodiments, the method of implanting further comprises drilling a borehole with a cannulated drill bit inserted around the guide wire. In certain embodiments, the method of implanting further comprises removing the drill bit and guide wire

In certain embodiments, the drill bit has a diameter smaller than the screw to be inserted. In certain embodiments, the drill bit is 3.2 mm in diameter.

In certain embodiments, implantation is percutaneous.

The disclosed method of implantation has the advantage of preventing overdrilling because the borehole needs only be drilled once rather than twice as in the conventional procedure. The bone harvesting features of the threads on the screw permit single drilling for insertion. Moreover, using insertion, the locking screw head engages and locks into the threaded plate hole. If necessary, the threaded plate hole also accepts nonlocking screws, which permit angulation. Tightening the screws “lag” the bone to the undersurface of the plate.

### Regenerative Medicine

“Regenerative medicine” refers to a branch of translational research in tissue engineering and molecular biology that deals with replacing, engineering, or regenerating human cells, tissues, or organs to restore or establish normal function. This field holds the promise of engineering damaged tissues and organs by stimulating the repair mechanisms within the patient's body to functionally heal previously irreparable tissues or organs. For example, during bone regeneration, new bone formation is primarily affected by physicochemical cues in the surrounding microenvironment. Tissue cells reside in a complex scaffold physiological microenvironment.

In certain embodiments, regenerative medicine is incorporated with the scaffolds or devices disclosed herein. Autogenous graft incorporation occurs in five stages: inflammation, vascularization, osteoinduction, osteoconduction, and remodeling.

Inflammation lasts for about 7 to 14 days. Initial insult to the local blood supply and decortications results in hematoma around the bone graft, in which inflammatory cells invade. The fibroblast-like cells in the inflammatory tissue transform into the fibrovascular stroma. Perioperative anti-inflammatory medications decrease fusion rates because of the inflammatory process.

Vascular buds appear in the fibrovascular stroma, resembling scar tissue formation during vascularization. Primary membranous bone forms near the decorticated bone. Next, minimal cartilage and endochondral ossification occur.

During osteoinduction at weeks 4 to 5, reparation comprises increased vascularization, necrotic tissue resorption, osteoblasts, and chondroblasts differentiation. In particular, stem cells differentiate into osteoblasts. New bone extends towards the central zone of the fusion mass. The cortical portion of the graft continues to resorb.

Osteoconduction is characterized by ingrowth into the host bone and creeping substitution. Osteoblasts create new bone while osteoclasts simultaneously resorb graft bone. A central zone of the endochondral interface is observed at the center of fusion mass, uniting the lower and upper halves of fusion. Pluripotent cells in this central zone differentiate into cartilaginous tissue with less vascularization.

During remodeling at weeks 6-10, a peripheral cortical rim forms around fusion. Bone marrow activity increases, forming secondary spongiosa. The cortical rim thickens. The trabecular process extends to the center of fusion. Remodeling typically completes one year after device implantation.

Pseudarthrosis (nonunion) was a leading cause of pain postoperatively and accounted for 45%-56% of revisions. Boney fusion directly correlates to successful clinical outcomes. Patients with pseudarthrosis were asymptomatic in about 30% of cases. Younger age has a significantly increased symptomatic pseudarthrosis rate (43.8 years vs. 52.1 years, p<0.01).

In certain embodiments, bone marrow aspirate (BMA) with allograft substitutes autogenous bone graft in single-level revision posterolateral lumbar fusion (PLF). In certain embodiments, bone marrow aspirate with allograft is more cost-effective than recombinant human bone morphogenetic protein-2 (rhBMP). In certain embodiments, bone marrow-derived cell-enriched allografts compare to autografts in bone grafting and spinal fusion procedures. In certain embodiments, BMA increases the regenerative potential of corticocancellous allogeneic bone grafts. When treating unicameral bone cysts, healing rates were high (98.7%) for bone marrow with demineralized bone matrix injection.

When introducing elements of the present disclosure or the embodiments(s) thereof, the articles “a,” “an,” “the,” and “said” are intended to mean that there are one or more of the elements. The terms “comprising,” “including,” and “having” are intended to be inclusive and mean that there may be additional elements other than the listed elements.

Having described the disclosure in detail, it will be apparent that modifications and variations are possible without departing from the scope of the disclosure defined in the appended claims.

Although the disclosure described herein is susceptible to various modifications and alternative iterations, specific embodiments thereof have been described in greater detail above. It should be understood, however, that the detailed description of the composition is not intended to limit the disclosure to the specific embodiments disclosed. Rather, it should be understood that the disclosure is intended to cover all modifications, equivalents, and alternatives falling within the spirit and scope of the disclosure as defined by the claim language.

Table 2 shows the reference numerals used in the figures.

TABLE 2
Reference numerals

| | |
|---|---|
| 100 | device |
| 200 | screw |
| 210 | proximal end |
| 215 | drive |
| 220 | distal tip |
| 230 | thread |
| 231 | leading edge |
| 232 | trailing edge |
| 235 | leading surface |
| 236 | trailing surface |
| 240 | core |
| 250 | openings |
| 251 | first opening |
| 252 | second opening |
| 260 | canula |
| 270 | cutting member |
| 280 | scaffold |
| 290 | narrow thread |
| 295 | shaft |
| 300 | bone |
| 310 | periosteum |
| 320 | cortical/compact bone |
| 330 | endosteum |
| 350 | medullary/spongy bone and marrow |
| 360 | borehole |
| 400 | adapter |
| 440 | tab |
| 450 | opening |
| 500 | cap/reduction cap |
| 510 | cap top |
| 520 | cap bottom |
| 530 | cap thread |
| 535 | external cap thread |
| 540 | cap body |
| 550 | castellation |
| 555 | divot |
| 560 | flange |
| 570 | collar |
| 571 | collar top |
| 572 | collar bottom |
| 576 | collar flange |
| 600 | guide wire |
| 700 | drill |
| 800 | headless driver |
| 900 | reduction driver |
| 950 | handle |

All references, patents, or applications, US or foreign, cited in the application are because of this incorporated by reference as if written herein in their entireties. Where any inconsistencies arise, the material disclosed herein controls.

From the preceding description, one skilled in the art can easily ascertain the essential characteristics of this invention and, without departing from the spirit and scope thereof, can make various changes and modifications of the invention to adapt it to various usages and conditions.

What is claimed is:

1. A reduction trauma screw, comprising a headless screw having narrow threads at the proximal end and a reduction cap adapted to couple to the headless screw via the narrow threads after the headless screw has been inserted into a substrate.

2. The screw of claim 1, wherein the substrate is a bone of a patient in need of reduction, traction, fixation, or combinations thereof.

3. The screw of claim 1 or 2, wherein the cap is spherical.

4. The screw of claim 1 or 2, wherein the cap is conical.

5. The screw of any one of claims 1 to 4, wherein the cap is flanged.

6. The screw of claim 5, wherein the cap comprise four flanges radially distributed at 90° intervals.

7. The screw of any one of claims 1 to 6, wherein the cap further comprises a collar configured to engage a substrate polyaxially.

8. The screw of claim 7, wherein the collar comprises ten flanges radially distributed at 36° intervals.

9. The screw of any one of claims 1 to 4, wherein the cap has a curved taper.

10. The screw of any one of claims 1 to 4, wherein the cap has a straight taper.

11. The screw of any one of claims 1 to 10, wherein the cap comprises external threads.

12. The screw of claim 1 or 2, wherein the cap is conical with a curved taper, conical with a straight taper, conical with a straight taper and external threads, or flanged with external threads.

13. The screw of any one of claims 1 to 12, wherein the cap comprises at least one castellation configured to be received by an adapter.

14. The screw of claim 13, wherein the cap comprises four castellations radially distributed at 90° intervals.

15. The screw of claim 13 or 14, wherein each castellation comprises a divot.

16. The screw of any one of claims 1 to 15, comprising a scaffold.

17. The screw of claim 16, wherein the scaffold is characterized by a randomized porosity pattern typical of a native trabecular bone comprising one or more structural cues that enhance at least one of multipotent mesenchymal stem cell (MSC) differentiation, osteoblast growth, extracellular matrix (ECM) deposition, and new bone formation.

18. The screw of claim 16 or 17, wherein the scaffold comprises a triply periodic minimal surface (TPMS) with a cubic repeating patten defining walls within the scaffold.

19. The screw of claim 18, wherein the TPMS is a Schwartz Diamond helically wrapped around a central axis of the screw to define a cubic repeating pattern in X/Y/Z dimensions for the scaffold.

20. The screw of claim 19, wherein the TPMS is helically wrapped into a single helix.

21. The screw of claim 19 or 20, having three radial spokes per turn of the helix.

22. The screw of any one of claims 19 to 21, wherein the cubic repeating pattern is about 1.8 mm in the X/Y/Z dimensions.

23. The screw of any one of claims 19 to 22, wherein the helical wrap of the screw is defined with a period three times the size of the cubic repeating pattern.

24. The screw of any one of claims 19 to 23, wherein the walls are about 0.5 mm thick.

25. The screw of claim 16 or 17, wherein the scaffold is a folded sheet scaffold.

26. The screw of claim 25, wherein the folded sheet scaffold is a shellular porous pseudorandom orientable architecture comprising a continuous folded sheet of topological genus-n, wherein real 3-dimensional space is divided into disjoint sub-volumes.

27. The screw of claim 26, wherein the sub-volumes are incongruent and non-intersecting.

28. The screw of claim 26 or 27, wherein the pseudorandom architecture is driven by a dimensionless 3-dimensional noise field.

29. The screw of any one of claims 26 to 28, wherein the sheet architecture has one or more characteristics chosen from continuous, perforated, functionally graded, semi-regular, and driven by modulating algorithms that control spatially-varying features.

30. The screw of any one of claims 16 to 29, wherein the threads of the screw near the proximal end are textured with a topography resembling the topography of the scaffold.

31. The screw of any one of claims 1 to 30, wherein the screw has reduced one or more screw loosening, screw backout, rod breakage, and lowered bone mineral density.

32. The screw of any one of claims 1 to 31, wherein the screw focuses bone growth throughout a shaft to minimize shear stresses on a distal tip and spreads micromotion throughout the screw to encourage bony ingrowth.

33. The screw of any one of claims 1 to 32, wherein the screw comprises at least one trephine to harvest bone internally within the screw.

34. A method of implanting a reduction trauma screw, comprising inserting a headless screw with a headless driver into a borehole in a bone, and coupling a reduction cap to the inserted headless screw via a reduction driver loaded with the reduction cap by using the headless driver to counter-torque against the reduction driver.

35. The method of claim 34, wherein the headless screw is the headless screw of any one of claims 1 to 33.

36. The method of claim 34 or 35, wherein the reduction cap is the reduction cap of any one of claims 1 to 16.

37. The method of any one of claims 34 to 36, wherein when the reduction cap is coupled to the headless screw, compression is provided to the bone without moving the headless screw.

38. The method of any one of claims 34 to 37, wherein, when present, the collar is adjusted polyaxially before compression is provided to the bone.

39. The method of any one of claims 34 to 38 further comprising aligning a drill guide, tissue protector, or both.

40. The method of any one of claims 34 to 39, further comprising inserting a guide wire down bone.

41. The method of claim 40 further comprising drilling a borehole with a cannulated drill bit inserted around the guide wire.

42. The method of claim 41 further comprising removing the drill bit and guide wire.

43. The method of claim 41 or 42, wherein the drill bit has a diameter smaller than the screw to be inserted.

44. The method of claim 43, wherein the drill bit is 3.2 mm in diameter.

45. The method of any one of claims 34 to 44, which is percutaneous.
